(19) United States
(12) Patent Application Publication
(10) Pub. No.: US 2023/0121506 A1
(43) Pub. Date: Apr. 20, 2023

(54) SYSTEM AND METHODS FOR EXAM SUGGESTIONS USING A DATABASE

(71) Applicant: GE Precision Healthcare LLC, Milwaukee, WI (US)

(72) Inventors: Svein Arne Aase, Trondheim (NO); Beate Ostensen, Horten (NO)

(21) Appl. No.: 17/451,465

(22) Filed: Oct. 19, 2021

(51) Int. Cl. G16H 50/20; G06T 7/00; G16H 30/20

(52) U.S. Cl. CPC G16H 50/20; G06T 7/0012; G16H 30/20; G06T 2200/24; G06T 2207/20092; G06T 2207/30004

(57) ABSTRACT

Methods and systems are provided for providing suggested diagnoses and/or measurements for a patient exam. In one example, a method for a user interface of a medical imaging system includes receiving a first user input from a user and determining a first measurement of a medical image based on the first user input, sending the first measurement and a value of the first measurement to a database of measurements, receiving, from the database, a second suggested measurement determined based on diagnosis tags including typical ranges including the value of the first measurement, and suggesting the second suggested measurement to the user before the user performs the second measurement.

### TECHNICAL FIELD

Embodiments of the subject matter disclosed herein relate to ultrasound imaging, and more particularly, to increasing an accurate diagnosis speed based on measurements taken on an ultrasound image.

### BACKGROUND

Medical ultrasound is an imaging modality that employs ultrasound waves to probe the internal structures of a body of a patient and produce a corresponding image. For example, an ultrasound probe comprising a plurality of transducer elements emits ultrasonic pulses which reflect or echo, refract, or are absorbed by structures in the body. The ultrasound probe then receives reflected echoes, which are processed into an image. Ultrasound images of the internal structures may be saved for later analysis by a clinician to aid in diagnosis and/or displayed on a display device in real time or near real time.

### SUMMARY

In one embodiment, a method for a user interface of a medical imaging system includes receiving a first user input from a user and determining a first measurement of a medical image based on the first user input, sending the first measurement and a value of the first measurement to a database of measurements, receiving, from the database, a second suggested measurement determined based on diagnosis tags including typical ranges including the value of the first measurement, and suggesting the second suggested measurement to the user before the user performs the second measurement.

The above advantages and other advantages, and features of the present description will be readily apparent from the following Detailed Description when taken alone or in connection with the accompanying drawings. It should be understood that the summary above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

Various aspects of this disclosure may be better understood upon reading the following detailed description and upon reference to the drawings in which:

FIG. 1 shows a block diagram of an exemplary embodiment of an ultrasound system;

FIG. 2 is a diagram showing an interface which forms part of the system of FIG. 1;

FIG. 3 is a flow chart illustrating an example method for generating a database;

FIG. 4 is a flow chart illustrating an example method for presenting a user with diagnosis tags and/or findings based on measurements taken during a current exam;

FIG. 5 is a flow chart illustrating an example method for qualifying a data source for an internal database;

FIG. 6 is a flow chart illustrating an example method for performing statistical calculations on measurements in the internal database;

FIG. 7 is a flow chart illustrating an example method for calculating p-values to determine a likelihood that an additional measurement may differentiate a diagnosis tag from other diagnosis tags during a patient exam;

FIG. 8 is a flow chart illustrating an example method for suggesting an additional measurement to take during a patient exam given a current list of possible diagnosis tags; and

FIG. 9 is a diagram showing a database that may be queried during a database lookup to determine potential diagnosis tags and/or measurements to suggest to the user.

### DETAILED DESCRIPTION

Some medical imaging systems, such as ultrasound systems, are relatively low cost, non-invasive, and easy to transport, use, and maintain. As such, these medical imaging systems are widely adopted globally. However, in many regions/markets, users of the medical imaging systems may not be experienced with respect to evaluating the images generated by the medical imaging system. For example, while it may be possible to use an ultrasound system to image a patient's heart in a remote or rural location that is far away from a large medical facility, often it may be difficult to find a cardiologist or other experienced and highly trained clinician to evaluate the images and make an accurate diagnosis. Further, even when trained clinicians are available to evaluate the images, some diagnoses may be complex and/or rare, which may result in the clinician having lower confidence in making an accurate diagnosis.

Thus, according to embodiments disclosed herein, possible diagnoses or findings may be automatically suggested based on measurements taken on medical images, such as ultrasound images. The suggested diagnoses/findings may be identified by interrogating a database of measurements that includes data from a plurality of prior patient exams, where the data from the plurality of prior exams includes a plurality of measurements of anatomical features, a plurality of diagnosis tags, and a respective statistical correlation between each measurement and diagnosis tag. In one example, the data from the plurality of prior patient exams includes calculated statistical p-values based on one or more measurements and associated measurement values in each exam and one or more diagnosis tags in each exam. Further, the database may also be interrogated to suggest one or more additional measurements that may be taken to increase a diagnosis confidence or differentiate between multiple possible diagnoses. Once the database has been populated with data and statistical calculations are performed, the structured results of the database may be saved in a format that uses a relatively small amount of memory and allows for simple lookups of similar exams, tags, and measurements to provide suggestions for diagnoses and measurements. In this way, once built and validated, the database may be saved and executed on a variety of devices, such as the medical imaging system itself, which may allow diagnoses and measurements to be suggested to users in a wide variety of clinical settings.

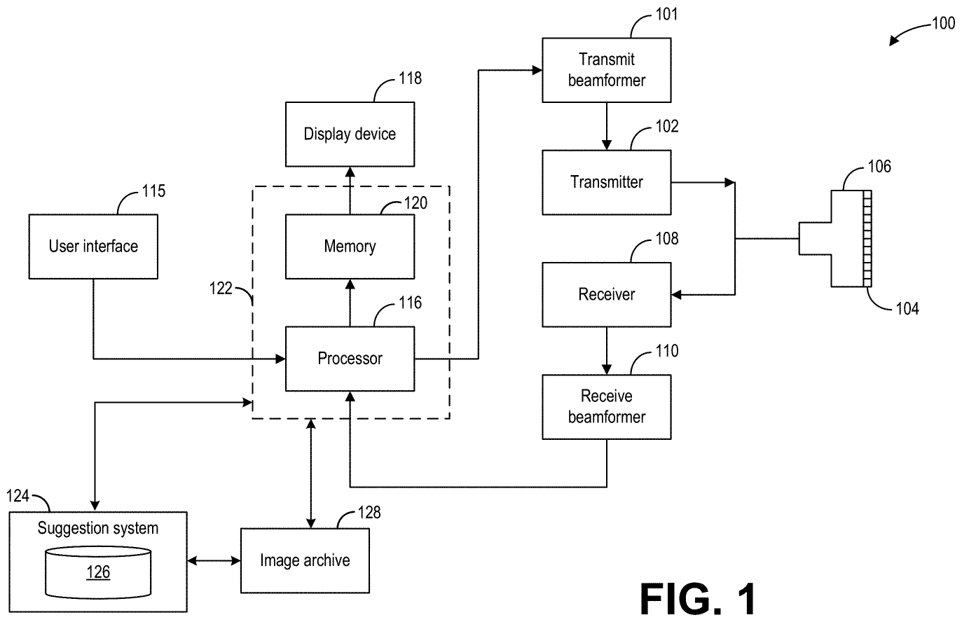

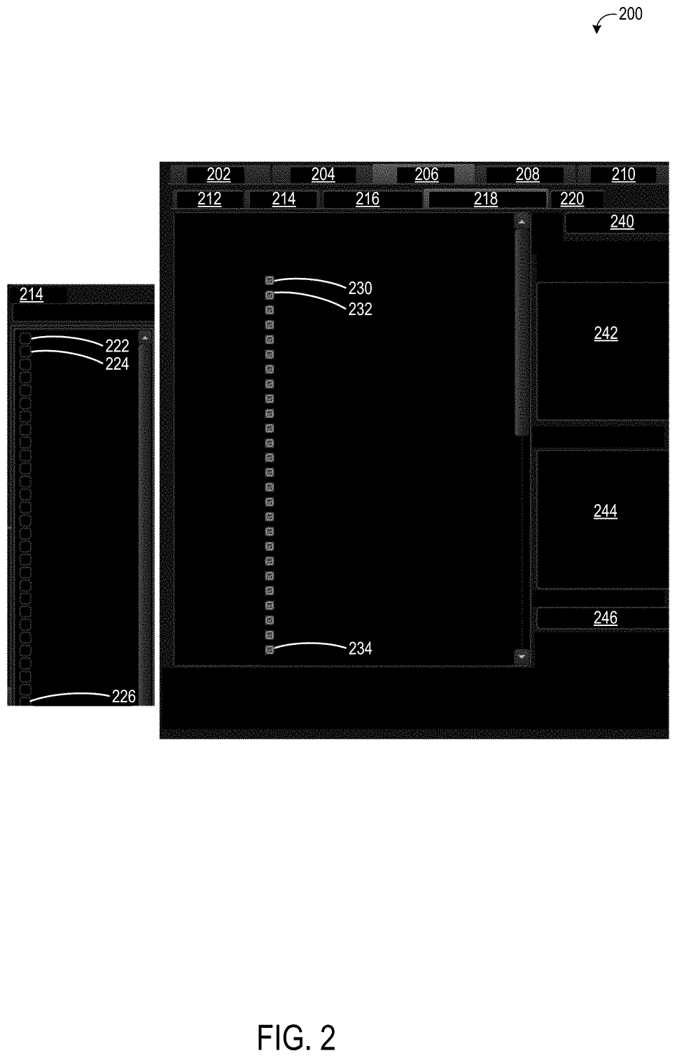

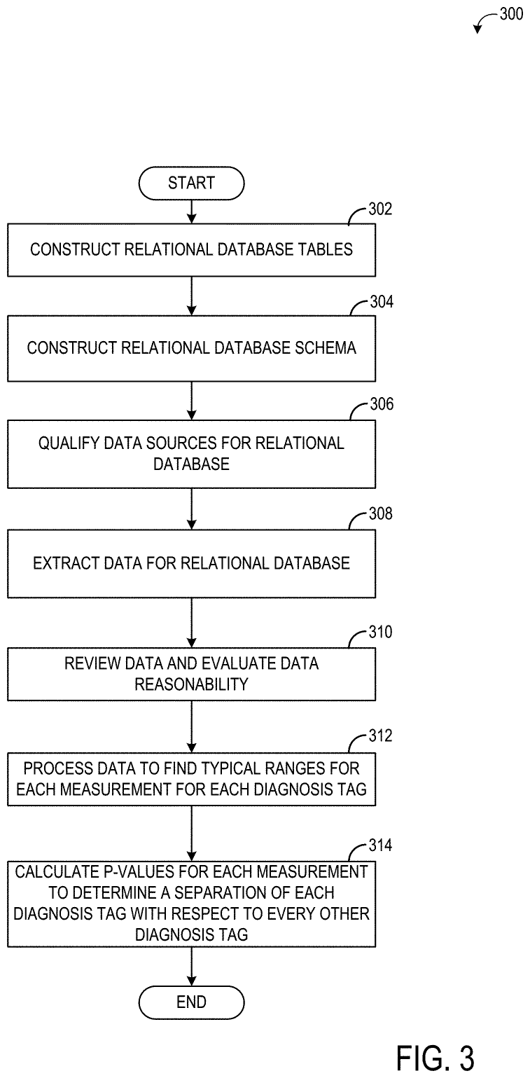

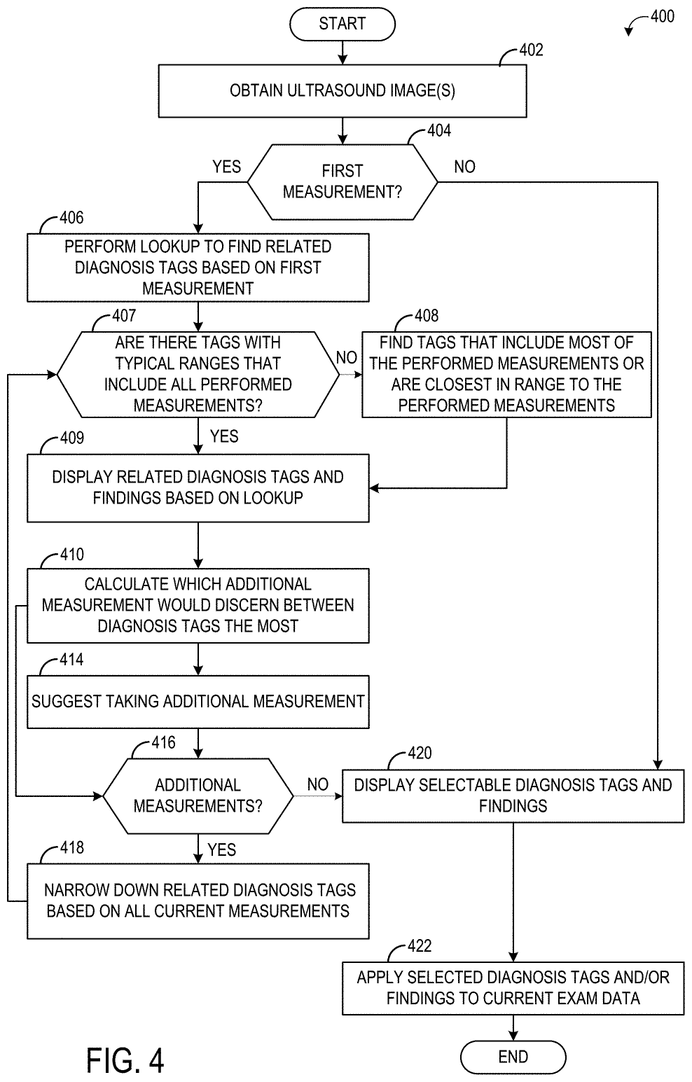

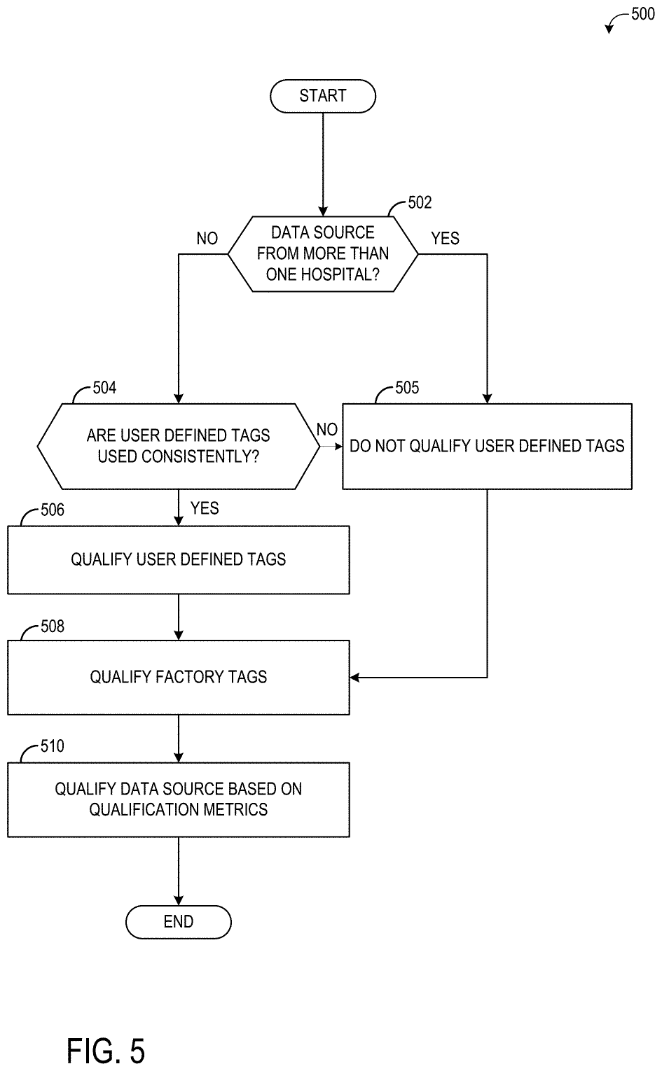

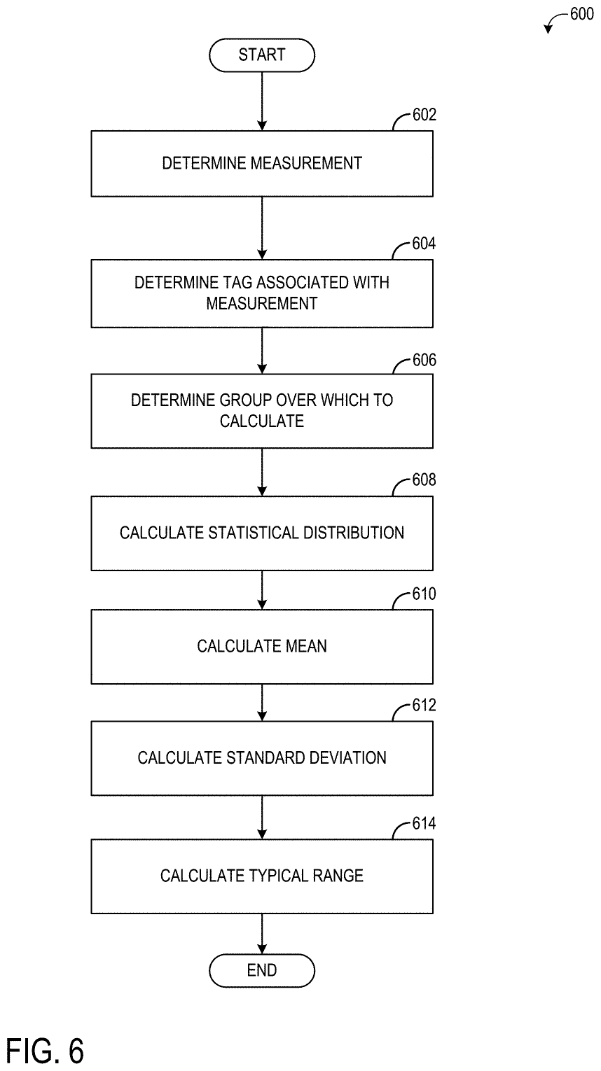

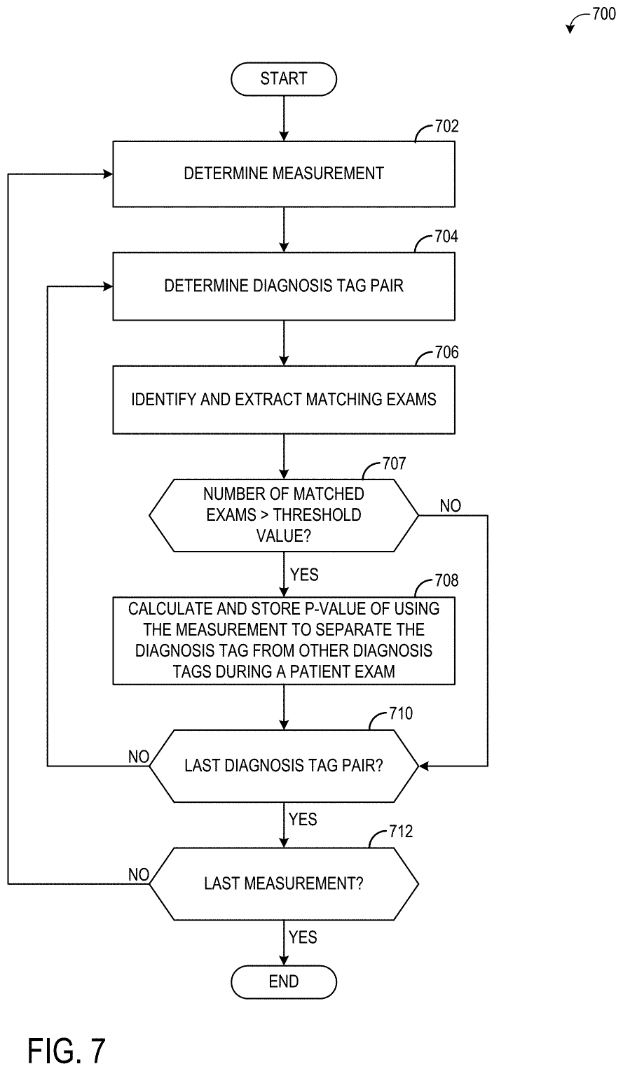

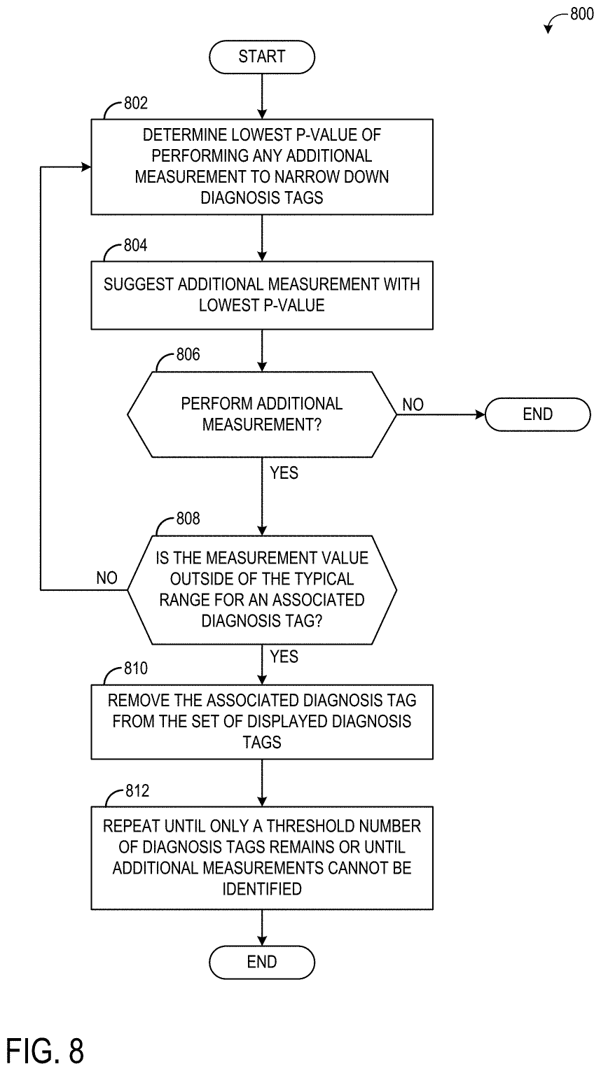

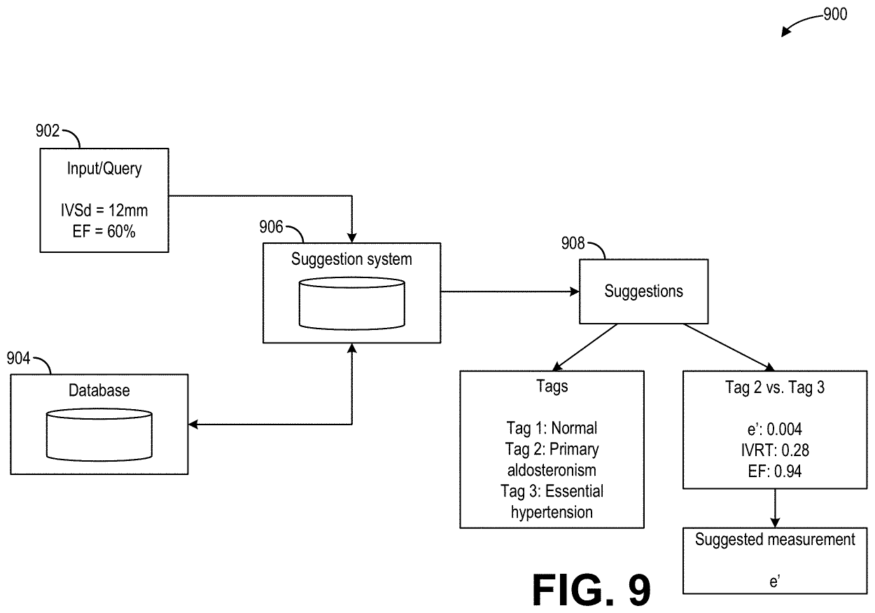

An example ultrasound system including an ultrasound probe, a display device, and an imaging processing system are shown in FIG. 1. Via the ultrasound probe, ultrasound images may be acquired and displayed on the display device. An interface displayed on a display device of FIG. 1 is shown in FIG. 2. A database including medical data may be generated and the data included in the database may be processed to have statistical calculations performed according to the method of FIG. 3. A user may apply diagnosis tags and/or findings tags to exam data for a current patient based on suggestions made via the database, according to the method of FIG. 4. When generating the database and/or including new data into the database, data sources may be qualified according to the method of FIG. 5. When processing data included in the database, statistical calculations may be performed according to the method of FIG. 6. Each measurement included in the database may have a calculated p-value for each associated diagnosis tag to determine a likelihood that performing the measurement during a patient exam may reduce a list of suggested diagnosis tags according to the method of FIG. 7. During a patient exam, a list of suggested diagnosis tags may be reduced by suggesting additional measurements according to the method of FIG. 8. An example database structure is shown in FIG. 9, illustrating potential calculations performed during a database lookup during user operation of the system.

Referring to FIG. 1, a schematic diagram of an ultrasound imaging system 100 in accordance with an embodiment of the disclosure is shown. The ultrasound imaging system 100 includes a transmit beamformer 101 and a transmitter 102 that drives elements (e.g., transducer elements) 104 within a transducer array, herein referred to as probe 106, to emit pulsed ultrasonic signals (referred to herein as transmit pulses) into a body (not shown). According to an embodiment, the probe 106 may be a one-dimensional transducer array probe. However, in some embodiments, the probe 106 may be a two-dimensional matrix transducer array probe. As explained further below, the transducer elements 104 may be comprised of a piezoelectric material. When a voltage is applied to a piezoelectric crystal, the crystal physically expands and contracts, emitting an ultrasonic wave. In this way, transducer elements 104 may convert electronic transmit signals into acoustic transmit beams.

After the elements 104 of the probe 106 emit pulsed ultrasonic signals into a body (of a patient), the pulsed ultrasonic signals reflect from structures within an interior of the body, like blood cells or muscular tissue, to produce echoes that return to the elements 104. The echoes are converted into electrical signals, or ultrasound data, by the elements 104 and the electrical signals are received by a receiver 108. The electrical signals representing the received echoes are passed through a receive beamformer 110 that outputs ultrasound data.

The echo signals produced by transmit operation reflect from structures located at successive ranges along the transmitted ultrasonic beam. The echo signals are sensed separately by each transducer element and a sample of the echo signal magnitude at a particular point in time represents the amount of reflection occurring at a specific range. Due to the differences in the propagation paths between a reflecting point P and each element, however, these echo signals are not detected simultaneously. Receiver 108 amplifies the separate echo signals, imparts a calculated receive time delay to each, and sums them to provide a single echo signal which approximately indicates the total ultrasonic energy reflected from point P located at range R along the ultrasonic beam oriented at angle θ.

The time delay of each receive channel continuously changes during reception of the echo to provide dynamic focusing of the received beam at the range R from which the echo signal is assumed to emanate based on an assumed sound speed for the medium.

Under direction of processor 116, the receiver 108 provides time delays during the scan such that steering of receiver 108 tracks the direction θ of the beam steered by the transmitter and samples the echo signals at a succession of ranges R so as to provide the proper time delays and phase shifts to dynamically focus at points P along the beam. Thus, each emission of an ultrasonic pulse waveform results in acquisition of a series of data points which represent the amount of reflected sound from a corresponding series of points P located along the ultrasonic beam.

According to some embodiments, the probe 106 may contain electronic circuitry to do all or part of the transmit beamforming and/or the receive beamforming. For example, all or part of the transmit beamformer 101, the transmitter 102, the receiver 108, and the receive beamformer 110 may be situated within the probe 106. The terms “scan” or “scanning” may also be used in this disclosure to refer to acquiring data through the process of transmitting and receiving ultrasonic signals. The term “data” may be used in this disclosure to refer to either one or more datasets acquired with an ultrasound imaging system. A user interface 115 may be used to control operation of the ultrasound imaging system 100, including to control the input of patient data (e.g., patient medical history), to change a scanning or display parameter, to initiate a probe repolarization sequence, and the like. The user interface 115 may include one or more of the following: a rotary element, a mouse, a keyboard, a trackball, hard keys linked to specific actions, soft keys that may be configured to control different functions, and a graphical user interface displayed on a display device 118.

The ultrasound imaging system 100 also includes a processor 116 to control the transmit beamformer 101, the transmitter 102, the receiver 108, and the receive beamformer 110. The processor 116 is in electronic communication (e.g., communicatively connected) with the probe 106. For purposes of this disclosure, the term “electronic communication” may be defined to include both wired and wireless communications. The processor 116 may control the probe 106 to acquire data according to instructions stored on a memory of the processor, and/or memory 120. The processor 116 controls which of the elements 104 are active and the shape of a beam emitted from the probe 106. The processor 116 is also in electronic communication with the display device 118, and the processor 116 may process the data (e.g., ultrasound data) into images for display on the display device 118. The processor 116 may include a central processor (CPU), according to an embodiment. According to other embodiments, the processor 116 may include other electronic components capable of carrying out processing functions, such as a digital signal processor, a field-programmable gate array (FPGA), or a graphic board. According to other embodiments, the processor 116 may include multiple electronic components capable of carrying out processing functions. For example, the processor 116 may include two or more electronic components selected from a list of electronic components including: a central processor, a digital signal processor, a field-programmable gate array, and a graphic board. According to another embodiment, the processor 116 may also include a complex demodulator (not shown) that demodulates the real RF data and generates complex data. In another embodiment, the demodulation can be carried out earlier in the processing chain. The processor 116 is adapted to perform one or more processing operations according to a plurality of selectable ultrasound modalities on the data. In one example, the data may be processed in real-time during a scanning session as the echo signals are received by receiver 108 and transmitted to processor 116. For the purposes of this disclosure, the term “real-time” is defined to include a procedure that is performed without any intentional delay. For example, an embodiment may acquire images at a real-time rate of 7-20 frames/sec. The ultrasound imaging system 100 may acquire 2D data of one or more planes at a significantly faster rate. However, it should be understood that the real-time frame-rate may be dependent on the length of time that it takes to acquire each frame of data for display. Accordingly, when acquiring a relatively large amount of data, the real-time frame-rate may be slower. Thus, some embodiments may have real-time frame-rates that are considerably faster than 20 frames/sec while other embodiments may have real-time frame-rates slower than 7 frames/sec. The data may be stored temporarily in a buffer (not shown) during a scanning session and processed in less than real-time in a live or off-line operation. Some embodiments of the invention may include multiple processors (not shown) to handle the processing tasks that are handled by processor 116 according to the exemplary embodiment described hereinabove. For example, a first processor may be utilized to demodulate and decimate the RF signal while a second processor may be used to further process the data, for example by augmenting the data as described further herein, prior to displaying an image. It should be appreciated that other embodiments may use a different arrangement of processors.

The ultrasound imaging system 100 may continuously acquire data at a frame-rate of, for example, 10 Hz to 30 Hz (e.g., 10 to 30 frames per second). Images generated from the data may be refreshed at a similar frame-rate on display device 118. Other embodiments may acquire and display data at different rates. For example, some embodiments may acquire data at a frame-rate of less than 10 Hz or greater than 30 Hz depending on the size of the frame and the intended application. A memory 120 is included for storing processed frames of acquired data. In an exemplary embodiment, the memory 120 is of sufficient capacity to store at least several seconds' worth of frames of ultrasound data. The frames of data are stored in a manner to facilitate retrieval thereof according to its order or time of acquisition. The memory 120 may comprise any known data storage medium.

In various embodiments of the present invention, data may be processed in different mode-related modules by the processor 116 (e.g., B-mode, Color Doppler, M-mode, Color M-mode, spectral Doppler, Elastography, TVI, strain, strain rate, and the like) to form 2D or 3D data. For example, one or more modules may generate B-mode, color Doppler, M-mode, color M-mode, spectral Doppler, Elastography, TVI, strain, strain rate, and combinations thereof, and the like. As one example, the one or more modules may process color Doppler data, which may include traditional color flow Doppler, power Doppler, HD flow, and the like. The image lines and/or frames are stored in memory and may include timing information indicating a time at which the image lines and/or frames were stored in memory. The modules may include, for example, a scan conversion module to perform scan conversion operations to convert the acquired images from beam space coordinates to display space coordinates. A video processor module may be provided that reads the acquired images from a memory and displays an image in real time while a procedure (e.g., ultrasound imaging) is being performed on a patient. The video processor module may include a separate image memory, and the ultrasound images may be written to the image memory in order to be read and displayed by display device 118.

In various embodiments of the present disclosure, one or more components of ultrasound imaging system 100 may be included in a portable, handheld ultrasound imaging device. For example, display device 118 and user interface 115 may be integrated into an exterior surface of the handheld ultrasound imaging device, which may further contain processor 116 and memory 120. Probe 106 may comprise a handheld probe in electronic communication with the handheld ultrasound imaging device to collect raw ultrasound data. Transmit beamformer 101, transmitter 102, receiver 108, and receive beamformer 110 may be included in the same or different portions of the ultrasound imaging system 100. For example, transmit beamformer 101, transmitter 102, receiver 108, and receive beamformer 110 may be included in the handheld ultrasound imaging device, the probe, and combinations thereof.

After performing a two-dimensional ultrasound scan, a block of data comprising scan lines and their samples is generated. After back-end filters are applied, a process known as scan conversion is performed to transform the two-dimensional data block into a displayable bitmap image with additional scan information such as depths, angles of each scan line, and so on. During scan conversion, an interpolation technique is applied to fill missing holes (i.e., pixels) in the resulting image. These missing pixels occur because each element of the two-dimensional block should typically cover many pixels in the resulting image. For example, in current ultrasound imaging systems, a bicubic interpolation is applied which leverages neighboring elements of the two-dimensional block. As a result, if the two-dimensional block is relatively small in comparison to the size of the bitmap image, the scan-converted image will include areas of poor or low resolution, especially for areas of greater depth.

The processor 116 and memory 120 may be included in a computing device 122. Computing device 122 may be a local device configured to be positioned in the same room/area as the probe 106 and may be coupled to the probe 106 via a wired or wireless connection. The computing device 122 may include a communication subsystem that may allow computing device 122 to communicate with additional external computing devices. As shown, computing device 122 is communicatively coupled to a suggestion system 124 and an image archive 128. Suggestion system 124 may be a computing device having resources (e.g., memory, processors) allocated to building and utilizing a database of measurements (referred to herein as database 126). As will be explained in more detail below, via the database 126, the suggestion system 124 may provide suggestions for diagnosis tags, findings, and/or additional measurements to be taken for a patient exam that includes medical images, such as ultrasound images generated by ultrasound system 100. The database 126 may be populated with data received from image archive 128, for example. Image archive 128 may be a picture archiving and communication system (PACS), a vendor neutral archive (VNA), or another suitable storage system configured to store patient exams. While not shown in FIG. 1, information stored on image archive 128 may be accessible through a separate computing device, referred to as a workstation, that may have a display device, user input devices, etc.

While FIG. 1 shows the ultrasound computing device (e.g., computing device 122), the suggestion system 124, and image archive 128 as separate devices, it is to be understood that in some examples, one or more of the devices may be combined in a single device. For example, the suggestion system 124 may reside on the image archive 128. Further, in some examples, the database 126 may be included as part of a separate device or the database 126 may be included as part of the image archive 128. In still further examples, aspects of suggestion system 124 may be included on computing device 122. For example, after database 126 has been built and prior patient exams have been processed to determine typical ranges for each of a plurality of measurements for each of a plurality of diagnosis tags as well as statistical correlations to differentiate diagnosis tags based on the measurement ranges and diagnosis tags (as explained below with respect to FIG. 3), the typical ranges and statistical correlations may be stored locally on the computing device 122 and the computing device 122 may be configured to provide suggestions for diagnosis tags, findings, and measurements based on the mapping.

Turning now to FIG. 2, it shows an embodiment of an interface 200 that may form part of the system of FIG. 1. In one example, the interface 200 may be displayed on a display device such as display device 118 of FIG. 1, or on a separate display device communicatively coupled to a storage device configured to save medical images, such as a PACS workstation. Interface 200 may display a plurality of diagnosis tags, findings, and/or tags to a user, allowing the user (e.g., a clinician) to select any amount of diagnosis tags, findings, and/or tags to be included as part of a current patient exam. As used herein, a patient exam may include one or more medical images of a patient, such as one or more ultrasound images, and associated diagnosis tags, findings, tags, and/or measurements that are selected, performed, or otherwise applied by a clinician. To complete the patient exam, the clinician may analyze the one or more medical images, perform measurements of anatomical features present within the one or more medical images, and use the interface 200 to associate diagnosis tags, findings, and/or tags with the one or more medical images, which may all be saved as part of the patient exam. A patient exam may also be referred to herein as a patient report.

Menu buttons, such as first menu button 202, second menu button 204, third menu button 206, fourth menu button 208, and fifth menu button 210, may represent selectable menus the user may choose when interacting with the system, labeled accordingly. A selected menu may be visually indicated by a color change, such as third menu button 206. In one example, third menu button 206 may be a menu for reports, where the user may view additional menus/submenus in order to select diagnosis tags, findings, etc., to be included in the report.

Submenu buttons, such as first submenu button 212, second submenu button 214, third submenu button 216, fourth submenu button 218, and fifth submenu button 220, may represent selectable submenus the user may choose when interacting with a selected menu of the system, labeled accordingly. A selected submenu may be visually indicated by a color change, such as fourth submenu button 218.

In one example, second submenu button 214 may be a submenu for diagnosis tags, where a list of available/selectable diagnosis tags may be displayed when the second submenu button 214 is selected. All diagnosis tags, including a first diagnosis tag 222, a second diagnosis tag 224, and an Nth diagnosis tag 226 may be displayed, where N may be a number of total diagnosis tags in the diagnosis tags submenu. If the user selects one of the diagnosis tags, that diagnosis tag may be saved as part of the patient exam/report. The diagnosis tags may include diseases, disorders, symptoms, or other clinically-relevant observations, and in some examples may be defined by national or international regulatory/governing bodies, such as ICD codes. In some examples, the user may specify the type of exam being conducted (e.g., an echocardiogram) via the interface 200, and a subset of possible diagnosis tags related to the exam type may be displayed.

In one example, fourth submenu button 218 may be a submenu for findings, where a list of findings may be displayed upon the fourth submenu button 218 being selected, allowing the user to look through finding tags that may be selected and applied to the report. All finding tags, including a first finding tag 230, a second finding tag 232, and an Mth finding tag 234 may be displayed, where M may be a number of total finding tags in the findings submenu. Findings may be similar to diagnosis tags and thus indicate diseases, disorders, symptoms, etc. Findings may be user-specified and/or hospital-specified and may include findings drawn from diagnosis tags as well as additional patient information, such as patient history. Similar to the diagnosis tags, the list of findings that is displayed may be based on the type of exam being performed.

In some examples, a user may be able to specify a new finding or include additional information about an existing finding by entering information into additional boxes, including a label box 240, a findings text box 242, a conclusion text box 244, and a billing code box 246. The user may enter input to label box 240 to define a display label (e.g., name) for a finding, where label 240 may display anywhere a findings tag may be displayed as a representation of the findings tag. Via the findings text box 242, the user may enter a detailed description of a findings tag, such that any information relating to, associated with, or further detailing a findings tag may be included. Via the conclusion text box 244, the user may enter guided diagnosis information regarding possible diagnoses or conclusions to make about the patient based on the medical images and patient history with the associated findings tag. In one example, information entered via the conclusion text 244 of a findings tag may include a plurality of diagnoses for the user to consider based on the information associated with the findings tag. Billing code 246 may include related billing codes to apply to the current patient exam based on the associated findings tags.

If the user chooses to add a user defined findings tag, the user may fill out label 240, findings text 242, conclusion text 244, and billing code 246 to apply the user defined findings tag to the system. Further, while not shown in FIG. 2, via interface 200, medical images may be displayed and measurements may be performed and saved via interface 200. For example, an image of a heart may be displayed and a user may measure the thickness of the interventricular septum (IVS) of the heart via one or more user inputs (e.g., the user may place a first measurement point on a first side of the IVS and place a second measurement point on a second side of the IVS and the thickness may be measured as the distance from the first point to the second point). These measurements may be saved as part of the patient exam/report.

Thus, interface 200 may be displayed during the analysis stage of a patient exam where medical images may be reviewed by a clinician such as a cardiologist to confirm or rule out one or more patient conditions, diseases, disorders, etc. In order to select a diagnosis tag or finding, the clinician may perform one or more measurements of anatomical features present in the medical images and choose one or more diagnosis tags and/or findings based on the measurements. For example, the patient exam may be an echocardiogram (also referred to herein as an echo) and the medical images may include a plurality of ultrasound images of the patient's heart, in various standard views, including Doppler imaging. The clinician may review the medical images and take measurements, such as distance measurements, area measurements, velocity measurements, etc., of various features of the heart, such as the left ventricle, right ventricle, interventricular septum, blood flow, etc. In certain exams such as echoes, the number of different measurements that may be taken is relatively large (20 or greater measurements performed, out of a larger number of possible measurements available to be taken, such as 100 possible measurements) and the number of different diagnosis tags and findings that may be available for selection may also be relatively large, such as 10 or more diagnosis tags and/or findings. Each clinician may choose to take different measurements and may draw different conclusions from the measurements. Further, some clinicians may rely on visual assessment rather than taking measurements.

Accordingly, the amount of time for performing a patient exam may be lengthy, and the lack of standardized protocols for performing the patient exam may result in inconsistent patient diagnoses, particularly by inexperienced users. The sheer volume of possible measurements that may be performed in echoes or other complex exams may present a challenge for inexperienced users, who may not be aware of which measurements may best indicate a given diagnosis, or which diagnosis to make given the large number of available measurements.

Thus, as described herein, during a patient exam where measurements of anatomical features present in medical images are taken in order to select one or more diagnosis tags and/or findings, suggestions may be provided for subsequent measurements and/or diagnosis tags/findings based on one or more prior measurements. The suggestions may be generated based on a database of processed measurements, such as database 126 of FIG. 1. The database of processed measurements may include measurements and diagnosis tags/findings from a plurality of prior patient exams that have been processed via statistical calculations performed by the system, such that each measurement may include an associated p-value for each pair of diagnosis tags and a typical range of measurement values for each diagnosis tag in the database, discussed in detail with respect to FIG. 6, based on the measurements performed in previous patient exams, the values of those measurements, and any diagnosis tags or findings tags associated with the exams.

FIG. 3 shows a flow chart illustrating an example method 300 for constructing a relational database of medical information and executing a plurality of statistical calculations on the medical information included in the database. Method 300 is described with regard to the systems and components of FIGS. 1-2, though it should be appreciated that the method 300 may be implemented with other systems and components without departing from the scope of the present disclosure. Method 300 may be carried out according to instructions stored in non-transitory memory of a computing device, such as memory 120 and processor 116. In other examples, method 300 may be carried out by a computing device having non-transitory memory and one or more processors and in communication with the ultrasound system of FIG. 1 and/or an image archive, such as suggestion system 124 of FIG. 1.

At 302, method 300 includes constructing relational database tables that will include data from a plurality of prior anonymized patient exams. The relational database tables may be an internal database (e.g., internal to a specific hospital or other medical facility) constructed according to guidelines a hospital or medical facility may adhere to, and thus at least in some examples the information from the plurality of prior patient exams included in the relational database may be extracted/obtained from only that hospital or medical facility. In other examples, the plurality of prior patient exams included in the relational database may be extracted/obtained from more than one hospital or medical facility. Database tables that may be constructed include an examination table, a measurement table, a diagnosis tag table, and a findings table, though it will be appreciated that any amount of database tables may be constructed to include relevant medical data as it relates to embodiments of this disclosure. The examination table may include identifying information for each of the plurality of prior patient exams (e.g., exam type, such as echocardiogram, fetal ultrasound, etc.). In one example, the measurements database table may include all measurements taken in each of the plurality of prior patient exams, including the values of each measurement, with possible associated information for each measurement including a unit of measurement, relevant patient demographic data (e.g., age, body mass index or body surface area, sex) and the like. The diagnosis tag table and findings table may each include the diagnosis tags and findings tags, respectively, from each of the plurality of prior patient exams. In some examples, the diagnosis tag table and the findings table may be combined into one table.

At 304, method 300 includes constructing relational database schema. In one example, the measurement database table may have a many to many relationship with the diagnosis tags database table, the measurement database table may have a many to many relationship with the findings database table, and the diagnosis tags database table may have a many to many relationship with the findings database table.

At 306, method 300 includes qualifying data sources for the relational database. Data (e.g., from one or more prior patient exams) may be acquired from external sources (e.g., other hospitals) to aggregate with the internal relational database. Qualifying data from external sources may include checking a consistency of user defined tags, such as user defined findings tags. For example, different hospitals may follow different standards/protocols for naming findings and thus some findings may have different names depending on the hospital from which the exam was obtained. In one example, if the data is acquired from one or more hospitals external to the internal hospital, user defined tags may be allowed to be included and aggregated with the relational database if the user defined tags are consistent with the nomenclature used in the internal hospital (e.g., originally included in the relational database). To determine if the user defined tags are consistent with the internal hospital nomenclature, each user defined tag from the external sources may be compared to the tags from the internal source (e.g., the internal image archive), and if a given tag from the external source matches a tag from the internal source, that tag may be determined to be consistent with the internal nomenclature. A similar approach may be taken to determine if user defined tags from the internal source are consistent with each other. For example, user defined tags that are recently-used tags (e.g., used within the past three months) may be identified as consistent and/or user defined tags that are frequently used tags (e.g., used more than 5 times) may be identified as consistent. In another example, if the data is acquired from more than one hospital, user defined tags may not be included and aggregated with the relational database due to a likelihood that user defined tags may not be consistent among the multiple hospitals. Rather, only tags that are known to be consistent (e.g., machine-based tags, ICD codes) may be used. Qualifying data sources based on a number of hospitals the data may be acquired from is further discussed with respect to FIG. 5. Qualification metrics may further include quantifying a number of patient exams that may be included, quantifying a number of patient exams with diagnosis tags, findings tags, and the like that may be included, quantifying a number of measurements taken per patient exam that may be included, and quantifying a number of patient exams that may be signed off (e.g., a user tagged a patient exam as complete) that may be included. In one example, patient exam data that may qualify to be included in the relational database may include patient exams with 4 or fewer associated diagnosis tags and/or 8 or fewer associated findings, patient exams with between 10 and 200 associated measurements, and patient exams tagged as complete by a user, meaning any patient exam data not meeting these qualifications may not be included in the relational database.

At 308, method 300 includes extracting the data for the relational database. The data may include a subset or all of the non-image data from the plurality of prior patient exams. Contents of any external database that may be acquired may be converted by a plurality of scripts to a serializable and transferrable representation. In this way, the database tables described above may be populated with the data from the plurality of prior patient exams.

At 310, method 300 includes reviewing the data and evaluating a reasonability of the data. A user (e.g., expert clinician) may perform an evaluation of quality control on the data being extracted to the relational database by reviewing the data and evaluating if the data is reasonable. Evaluating the data may include evaluating measurements and the reasonability of the measurement values for each patient exam, evaluating tags associated with measurements and the reasonability of the tag or set of tags being associated with measurements for each patient exam, and the like. Data reasonability may be determined by the user, such that the user may determine boundaries of measurement values that may be considered reasonable for a measurement, tags that may be considered reasonable for a measurement or a set of measurements, and the like. Data reasonability may also be predetermined, such that any boundary measurement values, tag associations, and the like may be determined prior to the extraction of the data, allowing the user to compare extracted data to predetermined metrics of data reasonability. Unreasonable data may include data with measurement values exceeding a maximum boundary value or not exceeding a minimum boundary value, data with a number of tag associations exceeding a maximum boundary value, and the like. Data that is determined to be unreasonable may be discarded manually by the user or automatically by the system, such that unreasonable data may not be included in the relational database. In one example, data may be extracted from a first hospital to a second hospital, where both hospitals operate on a same standard of data quality control, so a user from the second hospital may not fully evaluate all extracted data as a result of the extracted data already being evaluated by a user from the first hospital after an initial recording of the data.

At 312, method 300 includes processing the data to find typical ranges for each measurement for each diagnosis tag. A typical range may be considered a defined range of values for a measurement associated with a diagnosis tag based on a statistical value determined from all included values for the measurement stored in the database associated with the diagnosis tag. In an example, the typical range may be based on a mean value and a standard deviation calculated for all the included values for the measurement associated with the diagnosis tag. For example, for a given diagnosis tag (e.g., tag X), all measurements from all exams that include that diagnosis tag may be analyzed, and for each of those measurements, all measurement values may be processed to determine the mean and standard deviation in order to define the range for that measurement and diagnosis tag. In some examples, the typical range may be defined by a maximum value that includes the mean plus the standard deviation and a minimum value that includes the mean minus the standard deviation. In other examples, the maximum and minimum values may be based on the mean plus or minus a modified value of the standard deviation. For example, the typical range may include a minimum value of a coefficient and the standard deviation multiplied together and subtracted from the mean value. The typical range may include a maximum value of the coefficient and the standard deviation multiplied together and added to the mean value. For example, the coefficient may be 1.96 (Mean+−1.96*StDev), corresponding to the typical range containing 95% of the measured values (under the assumption of a normal distribution of the measured values), or be 2.576 (Mean+−2.576*StDec) corresponding to the typical range containing 99% of the measured values (under the assumption of a normal distribution of the measured values). Processing the data and calculating the typical ranges of the data may be automatically performed by the system, so the user may perform another data quality control evaluation to verify a reasonability for the calculated typical ranges for each measurement for each diagnosis tag. Typical ranges may be calculated on a plurality of levels of granularity, meaning that typical ranges may be calculated for a measurement associated with a diagnosis tag but with additional grouping conditions applied, including but not limited to all patients, only males, only females, only patients within an age range, and only patients of a certain ethnicity. At any level of granularity, calculating typical ranges for measurements associated with diagnosis tags includes a same process of calculating a statistical distribution, a mean value, a standard deviation, and a typical range for all measurement values of a measurement with an associated diagnosis tag satisfying any additional grouping conditions that may be included. The calculations are discussed further below with respect to FIG. 6.

At 314, method 300 includes calculating p-values for each measurement and for each pair of diagnosis tags to determine a separation of each diagnosis tag with respect to every other diagnosis tag based on that measurement. Each p-value calculated for a given measurement and diagnosis tag pair based on a measurement value of the given measurement may indicate if the given measurement for one of the associated diagnosis tags includes a measurement value that may be the same or significantly different than a measurement value for the given measurement for the other diagnosis tag of the pair. Because a p-value may be calculated (for the given measurement) for each possible pair of diagnosis tags, a p-value may be determined that indicates a contribution of that measurement to a level of differentiation between all tags. The calculated p-values may be represented in a data structure relating the given measurement associated with any included diagnosis tag in the system to an amount of separation each diagnosis tag included in the system may have from each other if the given measurement is taken during a patient exam.

In order to actually calculate p-values for a measurement's ability to discern between tags, the exams (and thus implicitly the patients) are first matched using matching criteria, and only exams that are relatively highly matched with the matching criteria are selected for the calculation of the p-values. The matching may be based on patient sex, age, blood pressure, body surface area (BSA), weight, height, and/or heart rate. For example, using patient sex and BSA to match exams prior to calculation of the p-values, for a given measurement m and a pair of diagnosis tags A and B, the goal of the analysis is to determine how well does measurement m discern between tags A and B. To accomplish this, all exams with measurement m and tag A are identified and extracted from the database (to form a database view_m_A). Likewise, all exams with measurement m and tag B are identified and extracted from the database (to form a database view_m_B). By using a matching optimization function (see below), the 50 pairs between view_m_A and view_m_B which have the best (highest numerical value) matching score are identified (or other suitable number of pairs, such as the 100 pairs with the highest matching score or the 40 pairs with the highest matching score).

The matching optimization function may assign points based on how well the exams match or do not match based on sex and BSA (in this example). For example, 0 points may be assigned when there is a sex match and −1 point may be assigned when there is a sex mismatch. For a BSA match, points may be assigned based on the equation −1*(Abs(BSA from view_m_A−BSA from view_m_B)/(BSA from view_m_B)). The matching optimization function may then equal the calculated sex match or mismatch value plus the BSA match value divided by two. This will have values [−1, 0]. It is to be appreciated that a similar approach may be taken for other matching criteria and that if more than two matching criteria are evaluated, the matching optimization function may include summing all the match values/points and dividing by the total number of criteria evaluated. Further, for age matching, points/values may be assigned based on whether or not exams match based on age ranges (e.g., 0 or −1 based on whether or not the exams were performed on patients in the same age range, which may be pediatric versus adult or more granular age ranges).

After this matching, a Pearson correlation between the two sets with respect to measurement m can be calculated to get a p-value. P-values may be calculated by performing independent t-tests, where the independent t-tests may determine if mean values from each set of measurement values associated with a diagnosis tag are significantly different from each other. Calculating the p-values is discussed further below with respect to FIG. 7. After calculations are performed for all pairs of tags and for all measurements associated with each pair of tags, p-values and any other included results from the calculations may be stored in a data structure which may be keyed by a pair of tags and ordered by p-values. In one example, the data structure may be ordered by p-values from a smallest p-value to a largest p-value, though other ways of organizing the data structure are possible without departing from the scope of this disclosure, such as ordering the p-values from largest to smallest.

FIG. 4 shows a flow chart illustrating an example method 400 for a user applying diagnosis tags and/or findings to patient exam data for a current patient based on measurements taken and related patient exam data from a database, such as the database of measurements 126 of FIG. 1 and/or the relational database described above with respect to FIG. 3. Method 400 may be carried out according to instructions stored in non-transitory memory of a computing device, such as memory 120 storing instructions executable by processor 116. In other examples, method 400 may be carried out by a computing device having non-transitory memory and one or more processors and in communication with the ultrasound system of FIG. 1 and/or an image archive and with the relational database, such as a PACS workstation or clinician device.

At 402, method 400 includes obtaining ultrasound images from a current patient exam. Ultrasound images may be acquired using an ultrasound system, such as the ultrasound imaging system of FIG. 1. In some examples, the ultrasound images may be acquired and displayed while method 400 is executed, such that measurements are taken and diagnosis tags and/or findings are selected for the exam at substantially the same time the images are acquired. In other examples, the ultrasound images may be obtained from an image archive (e.g., a PACS) after the imaging session with the patient is complete. The ultrasound images may be displayed on an exam interface, such as the interface 200 described above with respect to FIG. 2.

At 404, method 400 includes determining if a first measurement is taken. When the ultrasound image is displayed on the display device, the user may make a measurement of distance, velocity, area, volume, frequency, or the like of one or more anatomical features in the displayed ultrasound image(s), via a first user input received at the computing device. In one example, the user may look at the ultrasound image on the display device and not make a measurement, opting to make a diagnosis or add associated information on only visual analysis. If the first measurement is not taken, method 400 may continue to 420 to display selectable diagnosis tags and/or findings via the exam interface. In some examples, the selectable diagnosis tags and/or findings may be displayed at any time during the exam in response to a user request, or the selectable diagnosis tags and/or findings may be displayed along with the ultrasound image(s) on the exam interface for the entire duration of the exam.

If the first measurement is taken, at 406, method 400 performs a lookup to find related diagnosis tags based on the first measurement. Lookup operations may be performed on the data, described with respect to FIG. 9, where related diagnosis tags may be searched for over all data that may include the first measurement. Related diagnosis tags may include any diagnosis tags associated with measurements from previous patient exams with matching units (e.g., units of distance, velocity, area, volume, frequency). For example, if a prior patient exam includes a given diagnosis tag (e.g., tag x) and a given measurement (e.g., measurement b), the measurement may be associated with the diagnosis tag. Related diagnosis tags may be identified based on those diagnosis tags being associated with measurements including typical ranges that may include a measurement value of the first measurement. A diagnosis tag may be considered related if a measurement associated with the diagnosis tag includes a typical range that includes the measurement value of the given measurement with matching units (e.g., units of distance, velocity, area, volume, frequency). In one example, a user may be a specialist specializing in one specific area of the body, so related diagnosis tags may include any diagnosis tags including measurements taken by the user from previous patient exams.

For example, the first measurement (including the type of measurement and value of the first measurement) may be sent to a database of measurements, such as the database 126 and/or the exam data of the relational database described above with respect to FIG. 3. A search may be performed to identify a list of probable diagnosis tags and/or findings based on the first measurement, where each probable diagnosis tag and/or finding is associated with a measurement that matches the first measurement and has a typical range that spans the value of the first measurement. The list of probable diagnosis tags may be identified without clustering any data (e.g., identified from the data of the relational database, where the data is not subject to any clustering algorithms). Clustering may include grouping the data in the relational database based on grouping parameters (e.g., measurement values, diagnosis codes), where data with similarities in groups may be considered more similar to other data in a same group than data in other groups based on the grouping parameters. In one example, the first measurement (and any prior measurements) may be used to identify equivalent measurements from the database without executing any clustering algorithms (e.g., on the data in the relational database).

It is to be appreciated that to identify a diagnosis tag with high confidence, more than one measurement may be required. Thus, the process described above (e.g., identifying probable tags based on the first measurement) may be iteratively repeated each time a new measurement is taken, and one or more probable tags may be identified once sufficient identifying/differentiating measurements have been taken and sent to the database of measurements. In other examples, the first measurement and any prior or subsequent measurements may be sent to the database of measurements in response to a user request for suggested diagnosis tags, findings, and/or further measurements, or the first measurement and any prior or subsequent measurements may be sent to the database of measurements in response to an indication that the current exam is complete (e.g., the user indicates that all measurements have been taken). Further, it is to be appreciated that the diagnosis tags may include a “normal” tag, such that measurements with values that fall within a normal range for that measurement may trigger selection of the normal tag.

At 407, method 400 includes determining if there are diagnosis tags with typical ranges that include all measurements performed during the current patient exam. For example, if five total measurements have been performed in the current exam, the list of probable diagnosis tags may only include diagnosis tags that are associated with all five measurements, and at least in some examples, include respective typical ranges for the measurements that span the respective measurement value for that measurement taken during the current exam. Determining if all measurement values included in the current patient exam are included in typical ranges for measurements associated with diagnosis tags may lead to more accurate diagnosis tags being associated with the current patient exam based on the diagnosis tag suggestions. If there are tags with typical ranges that include all measurements performed during the current patient exam, method 400 may proceed to 409, which includes displaying related diagnosis tags and findings based on the database lookup.

If there are no tags with typical ranges that include all measurements performed during the current patient exam, method 400 may proceed to 408, which includes finding diagnosis tags that include most of the measurement performed during the current patient exam or include typical ranges that are closest in range to the measurement values of the measurements performed during the current patient exam. In one example, if only one measurement is performed during the current patient exam and there are no diagnosis tags with typical ranges that include the measurement performed during the current patient exam, diagnosis tags including typical ranges closest in range to the measurement performed during the current patient exam may be found, where a threshold absolute value of difference may be used to determine if the measurement performed during the current patient exam is close enough to a typical range of a diagnosis tag to be considered related to the current patient exam.

At 409, method 400 includes displaying the related diagnosis tags and findings based on the lookup (e.g., the list of probable diagnosis tags discussed above). Diagnosis tags and findings may be displayed in an interface on a display device, such as interface 200 of FIG. 2. In one example, the display device may be the same display device used to display the ultrasound images. In another example, a secondary display device may be used to display the interface of diagnosis tags and findings. In this way, the result of the lookup/search of the first measurement may be a list of the tags which most closely match the measurement and measurement value of the first measurement, which may then be presented to the user. In some examples, each diagnosis tag that is displayed may be selectable or include an adjacent selectable element, such that the user may select a diagnosis tag from the displayed list to include as part of the current exam, which may expedite selection of diagnosis tags and reduce the amount of scrolling and user inputs required for the user to select diagnosis tags.

At 410, method 400 includes calculating which additional measurement would discern between diagnosis tags the most. For example, if more than one probable diagnosis tag is identified, one or more additional measurements may be suggested to assist the user in narrowing down and/or selecting a diagnosis code. The suggested measurements may be selected based on which measurements have a high likelihood of differentiating between diagnosis tags, which may be based on the p-values described above. As described above, a p-value may be calculated for each measurement and each pair of diagnosis tags. If the list of probable diagnosis tags includes two or more tags, a measurement may be suggested to differentiate between two of the diagnosis tags (e.g., a pair of diagnosis tags), based on the p-values of each measurement calculated for that pair of diagnosis tags. For example, if the list of probable diagnosis tags includes tag x and tag y, the data structure including the p-values described above may be queried to identify the p-values calculated for all measurements for the diagnosis tag pair of tag x, tag y, and the measurement having the lowest p-value may be selected and suggested to the user. If more than two diagnosis tags are included in the list of probable diagnosis tags, a measurement may be suggested to differentiate between diagnosis tags in each pair of diagnosis tags (e.g., if three diagnosis tags are suggested, three measurements may be suggested), or only the measurement with the lowest p-value may be suggested. Discerning between diagnosis tags may be considered a narrowing down of a current list of suggested diagnosis tags based on the p-values of each measurement associated with a diagnosis tag included in the current list of suggested diagnosis tags.

In some examples, based on the current measurement and related diagnosis tags found by the lookup, a calculation may be performed to determine if one or more additional measurements would increase a diagnosis confidence by discerning between diagnosis tags. If a plurality of additional measurements may be determined to increase a diagnosis confidence by discerning between diagnosis tags, the calculation may determine a minimum amount of additional measurements that may increase diagnosis confidence. In one example, the diagnosis confidence may be considered to be sufficiently increased if an amount of suggested diagnosis tags and findings is reduced by a predetermined amount or percentage. In another example, the diagnosis confidence may be considered to be sufficiently increased if an amount of increase in the diagnosis confidence is greater than a predetermined confidence threshold. The calculation of whether the additional measurement(s) would increase a diagnosis confidence may be performed by a separate computing device, e.g., the computing device storing the database of measurements and/or in communication with the database of measurements, such as the suggestion system 124, at least in some examples.

At 414, method 400 includes suggesting taking an additional measurement(s) based on the calculations from 410. The suggested additional measurements may be output for display on the display device (e.g., on the interface 200). The additional measurements may be suggested to the user before the user takes the additional measurements. When a measurement is suggested to the user, the measurement may be displayed (e.g., on the interface 200) as or adjacent to a selectable link/control button that, when selected, launches a measurement menu, a set of measurement control buttons, or other graphical user interface items that allow the measurement to be taken. In doing so, the user may take the suggested measurement without having to perform unnecessary scrolling or navigation through menus.

At 416, method 400 includes determining if additional measurements are taken. A user may optionally choose to make additional measurements based on user preference or possible suggestions from 414. The determination of whether additional measurements are taken may be based on received user input (e.g., user input placing measurement points). If additional measurements are not taken, method 400 may continue to 420, which includes displaying selectable diagnosis tags and findings.

If additional measurements are taken, at 418, method 400 includes narrowing down related diagnosis tags based on all current measurements taken. The same criteria adhered to by the lookup may be adhered to when narrowing down the related diagnosis tags. For example, the first measurement and the additional measurement(s) that are taken (including the type of measurements and values of the measurements) may be sent to the database of measurements, where a search is again performed to identify one or more probable diagnosis tags based on the first measurement and additional measurement(s). When more than one probable diagnosis tag is identified, further additional measurements may be suggested, as explained above. As more and more measurements are taken, the list of probable diagnosis tags or findings may be narrowed down. For example, after the first measurement, a first list of tags (each indicating a diagnosis tag or finding) may be returned to the user, where the first list of tags includes a subset of a plurality of possible tags and excludes remaining tags from the plurality of possible tags. After a second measurement is taken, a second list of tags may be returned to the user, where the second list of tags includes one or more tags from the first list of tags and excludes remaining tags from the first list of tags. In some examples, when the second measurement is received, only tags from the list of tags may be queried, which may reduce the processing power necessary to identify the second list of tags and lower the amount of time needed to identify the second list of tags.

Thus, upon performing the new search with the additional measurements, method 400 continues back to 407 to determine if there are diagnosis tags with typical ranges that include all currently performed measurements. This process of suggesting measurements and diagnosis tags may be iteratively repeated as more measurements are taken. For example, after the additional measurements are suggested, a second user input may be received from the user and a second measurement of the ultrasound image may be determined based on the second user input. The second measurement and a value of the second measurement may be sent to the database of measurements. A second list of probable diagnosis tags may be generated based on the first measurement, the value of the first measurement, the second measurement, and the value of the second measurement, based on the values of the first and second measurements being included in the typical ranges for the first measurement and second measurement, respectively, for the diagnosis tags. A third suggested measurement may be received from the database, where the third suggested measurement is determined based on the p-values calculated for each pair of diagnosis tags of the second list of probable diagnosis codes. The third suggested measurement may be suggested to the user before the user performs the third measurement. Further, it will be appreciated that the measurements that are taken and suggested, as well as the suggested diagnosis tags and findings, may apply to the currently displayed ultrasound image and/or other ultrasound images in the current patient exam. For example, echocardiograms may typically include a plurality of images taken at different anatomical views (e.g., PLAX, PSAX, A4C, etc.), with each view imaged in one or more imaging modes (e.g., B mode, M mode, Doppler, etc.). Thus, different measurements may be performed on different images, and as such, the ultrasound image that is currently displayed may change as method 400 progresses. Further, when method 400 is executed during an active imaging session, in addition or alternative to suggesting additional measurements, additional views and/or imaging modes may also be suggested to ensure a complete exam is performed, which may be guided by the measurements already taken.

Returning to 416, if additional measurements are not taken and/or if the user indicates that all measurements are complete, method 400 proceeds to 420, where method 400 includes displaying selectable diagnosis tags and findings. The displayed list of diagnosis tags and findings may be all possible diagnosis tags and findings for the type of exam being performed. In other examples, the displayed diagnosis tags and findings may be narrowed down based on the measurements that have been taken and suggested diagnosis tags/findings determined based on the measurements. From the displayed list of selectable diagnosis tags and findings, the user may select one or more diagnosis tags and/or findings to be saved as part of the patient exam, which may be influenced by the suggested diagnosis tags and/or findings.

At 422, method 400 includes applying selected diagnosis tags and/or findings to current patient exam data (e.g., to the current patient exam). The user may select any diagnosis tags and/or findings to apply the associated data with the current patient exam data. The user may also choose to apply user defined tags based on the current patient exam or user preference.

FIG. 5 shows a flow chart illustrating an example method 500 for qualifying data sources when extracting data from a data source for an internal relational database. In one example, method 500 may be executed during method 300 of FIG. 3, specifically during 306, when qualifying data sources for the internal relational database. Data (e.g., from one or more prior patient exams) may be acquired from external sources (e.g., other hospitals) to aggregate with the internal relational database. Qualifying data from external sources may include checking a consistency of user defined tags, such as user defined findings tags.

At 502, method 500 includes determining if an incoming data source is from more than one hospital. Different hospitals may follow different standards/protocols for naming findings and thus some findings may have different names depending on the hospital from which the exam was obtained.

If the incoming data source is from more than one hospital, method 500 may proceed to 505, which includes not qualifying user defined tags. User defined tags may be considered qualified if the user and/or the system chooses to include the user defined tags in the internal relational database, such that user defined tags that may not be qualified may not be included in the internal relational database. User defined tags may not be qualified when data sources come from more than one hospital due to a higher likelihood of inconsistency in user defined tags between the plurality of hospitals. In one example, a first user at a first hospital may use a user defined tag following different rules than a second user at a second hospital using the same user defined tag.

If the incoming data source is not from more than one hospital, method 500 may proceed to 504, which includes determining if user defined diagnosis tags are used consistently. In one example, if the data is acquired from one hospital to be included in the internal relational database, user defined tags may be allowed to be included and aggregated with the relational database if the user defined tags are consistent with the nomenclature used in the internal relational database (e.g., originally included in the relational database). To determine if the user defined tags are consistent with the internal relational database nomenclature, each user defined tag from the external source may be compared to the tags from the internal source (e.g., the internal image archive), and if a given tag from the external source matches a tag from the internal source, that tag may be determined to be consistent with the internal relational database nomenclature. A similar approach may be taken to determine if user defined tags from the internal source are consistent with each other. For example, user defined tags that are recently-used tags (e.g., used within the past three months) may be identified as consistent and/or user defined tags that are frequently used tags (e.g., used more than 5 times) may be identified as consistent. If user defined tags are not used consistently, method 500 may proceed to 505, which includes not qualifying user defined tags.

If user defined tags are used consistently, method 500 may proceed to 506, which includes qualifying user defined tags. After the two determinations, user defined tags that are known to be consistent may be qualified. Qualification of user defined tags may include choosing data (e.g., measurements, prior patient exams) that may proceed to a next step of qualifying the data source.

At 508, method 500 includes qualifying factory tags. Factory tags (e.g., machine-based tags, ICD codes) may be considered universally consistent among all embodiments of the systems of this disclosure. Qualification of factory tags may include marking data (e.g., measurements, prior patient exams) that may proceed to a next step of qualifying the data source.

At 510, method 500 includes qualifying the data source based on qualification metrics. Qualification metrics may include quantifying a number of prior patient exams that may be included, quantifying a number of prior patient exams with diagnosis tags, findings tags, and the like that may be included, quantifying a number of measurements taken per patient exam that may be included, and quantifying a number of patient exams that may be signed off (e.g., a user tagged a patient exam as complete) that may be included. In one example, patient exam data that may qualify to be included in the relational database may include patient exams with 4 or fewer associated diagnosis tags and/or 8 or fewer associated findings, patient exams with between 10 and 200 associated measurements, and patient exams tagged as complete by a user, meaning any patient exam data not meeting these qualifications may not be included in the relational database.

FIG. 6 shows a flow chart illustrating a method 600 for calculating a typical range for each measurement for each diagnosis tag based on the data in the relational database. In one example, method 600 may be executed during method 300 of FIG. 3, specifically at 312. Data may be considered to be reasonable in method 600 by qualification metrics described above in method 300 with respect to FIG. 3. A typical range may be considered a range of values for a measurement associated with a diagnosis tag, based on a mean value and a standard deviation calculated for each measurement and all the included values for the measurement associated with the diagnosis tag.

At 602, method 600 includes determining (e.g., selecting) a measurement from all measurements listed in the relational database. A typical range is calculated for each measurement in the database, and the measurements may be chosen in an iterative pattern, such that the process described herein may be performed iteratively on each measurement. In one example, a measurement may not have any associated tags, so a typical range may not be calculated for that measurement and the measurement may not be included in the iterative pattern.

At 604, method 600 includes determining a tag associated with the measurement. As explained above, a diagnosis tag may be associated with a measurement if the diagnosis tag and measurement are included in the same prior patient exam. A measurement may have a plurality of associated diagnosis tags, such that one measurement may have a plurality of associated typical ranges, with one typical range for each associated diagnosis tag.

At 606, method 600 includes determining a group over which to calculate a typical range. Typical ranges may be calculated on a plurality of levels of granularity, meaning that typical ranges may be calculated for a measurement associated with a diagnosis tag for all patients, but additional grouping conditions may also be applied to calculate a typical range for the measurement and the associated diagnosis tag as well as the specific grouping, meaning that one measurement may have a plurality of associated typical ranges for each diagnosis tag including any combination of further grouping conditions. Grouping conditions may include all patients, only males, only females, only patients within an age range, only patients of a certain ethnicity, and/or the like.

At 608, method 600 includes obtaining all the measurement values for the measurement that are included in exams having the selected diagnosis tag and the selected grouping conditions (e.g., all patients, or only males, only females, etc.) from the relational database and calculating a statistical distribution of the measurement values satisfying the previously determined parameters described above (e.g., measurement, diagnosis tag, grouping(s)). The statistical distribution may mathematically describe a probability that a measurement associated with a diagnosis tag may include an outcome value.

At 610, method 600 includes calculating a mean value of the measurement values satisfying the previously determined parameters (e.g., measurement, diagnosis tag, grouping(s)). The mean value may be an arithmetic average of all the measurement values satisfying the previously determined parameters, and the mean value may be used as a median value for a calculated typical range for the measurement, the associated tag, and any associated grouping parameters.

At 612, method 600 includes calculating a standard deviation of the measurement values satisfying the previously determined parameters (e.g., measurement, diagnosis tag, grouping(s)). The standard deviation may be a square root taken of how far the measurement values are spread out from the mean value, and the standard deviation may be used as a variable for a calculated typical range for the measurement, the associated tag, and any associated grouping parameters.

At 614, method 600 includes calculating a typical range of the measurement values satisfying the previously determined parameters (e.g., measurement, diagnosis tag, grouping(s)). The typical range may be bounded by a minimum value and a maximum value. The minimum value of the typical range may be a product of a coefficient and the standard deviation multiplied together and subtracted from the mean value. The maximum value of the typical range may be the product of the coefficient and the standard deviation multiplied together and added to the mean value. In this way, the typical range may be selected to include 95%-99% of all measurement values assuming a normal distribution. However, other mechanisms for determining the typical ranges are possible, such using a median value or a mode value rather than a mean value.

Processing the data and calculating the typical ranges of the measurement values may be automatically and/or iteratively performed by the system, so the user may perform another data quality control evaluation to verify a reasonability for the calculated typical ranges for each measurement for each diagnosis tag. At any level of granularity, calculating typical ranges for measurements associated with diagnosis tags includes a process of calculating a statistical distribution, a mean value, a standard deviation, and a typical range for all measurement values of a measurement with an associated diagnosis tag satisfying any additional grouping conditions that may be included. As such, this process may be repeated for each diagnosis tag and grouping condition for the selected measurement, and then repeated for the next measurement.

FIG. 7 shows a flow chart illustrating a method 700 for calculating a p-value to determine an amount of separation of a diagnosis tag with respect to another diagnosis tag in the system. Determining separation of diagnosis tags may facilitate more efficient measurement suggestions during patient exams. Calculated p-values may be used to determine a measurement may be suggested to a user during a patient exam given a current set of measurements and a current set of diagnosis tags. P-values may be calculated in an iterative pattern, meaning that method 700 may be executed in an iterative pattern in order to calculate p-values for each pair of diagnosis tags for each measurement. In one example, method 700 may be executed during method 300 of FIG. 3, specifically at 314. The calculated p-values may be represented in a data structure.

At 702, method 700 includes determining (e.g., selecting) a measurement from all the measurements in the relational database. Measurements may be chosen in an iterative pattern, such that p-values may be calculated for all measurements included in the system in the iterative pattern.

At 704, method 700 includes determining (e.g., selecting) a diagnosis tag pair from the diagnosis tags associated with the selected measurement (e.g., where the measurement and the diagnosis tag are included in at least one common exam). Diagnosis tag pairs may be chosen in an iterative pattern, such that p-values may be calculated for all diagnosis tag pairs included in the system in the iterative pattern. In one example, method 700 may be executed in an iterative pattern such that a plurality of p-values may be calculated for each measurement, where a p-value may be calculated for every diagnosis tag pair included in the system associated with that measurement.

At 706, method 700 includes identifying and extracting matching exams. P-values may be calculated on a plurality of levels of granularity, meaning that p-values may be calculated for a measurement and a diagnosis tag for all patients, but additional grouping conditions may also be applied to calculate a p-value for the measurement and the diagnosis tag pair as well as a specific grouping, meaning that one measurement may have a plurality of associated p-values for each diagnosis tag pair including any combination of further grouping conditions. Grouping conditions may include all patients, only males, only females, only patients within an age range, only patients of a certain ethnicity, and/or the like. Thus, as explained previously, exams having the selected measurement and the first diagnosis tag of the pair may be extracted to form a first group of exams, exams having the selected measurement and the second diagnosis tag of the pair may be extracted to form a second group of exams, and a set of matching exams may be formed by calculating a matching score for each pair of exams (e.g., where each pair includes an exam from the first group and an exam from the second group) and selecting the 50 or 100 pairs with the highest matching scores. The matching scores may be based on selected matching criteria, such as based on age, gender, BSA, etc. To calculate p-values for different groupings, different matching criteria may be applied.

At 707, method 700 includes determining if the number of matched exams is greater than a threshold value. The threshold value may be a predetermined number calculated by the system or determined by a user to ensure a sufficient amount of database entries including the measurement and the diagnosis tags and that sufficient match based on the matching criteria exist in order to calculate a p-value with high accuracy. In one example, the threshold value may be 50. If the number of matched exams is not greater than the threshold value, method 700 may proceed to 710 to determine if the diagnosis tag pair is the last diagnosis tag pair in order to continue iteratively choosing measurements and diagnosis tag pairs for p-value calculation, which is explained in more detail below.

If the number of matched exams is greater than the threshold value, method 700 may proceed to 708, which includes calculating the p-value of using the measurement to separate the diagnosis tags of the diagnosis tag pair. A p-value may be calculated for each pair of diagnosis tags, such that each p-value may indicate a level of statistical difference between two diagnostic tags based on the typical ranges for the measurement for each of the two diagnosis tags. P-values may be calculated by performing independent t-tests on the extracted matched exams, where the independent t-tests may determine if the measurement values for the selected measurement of the two diagnosis tags are significantly different from each other.

At 710, method 700 determines if the current diagnosis tag pair is the last diagnosis tag pair for the measurement, of if p-values still need to be calculated for remaining diagnosis tag pairs. If the current diagnosis tag pair is not the last pair, method 700 loops back to 704 to select the next diagnosis tag pair and calculate the p-value for that diagnosis tag pair and the current measurement. If the current diagnosis tag pair is the last diagnosis pair (e.g., a p-value has been calculated for each diagnosis tag pair for the current measurement), method 700 proceeds to 712 to determine if the current measurement is the last measurement (e.g., for calculating p-values). If the current measurement is not the last measurement (e.g., p-values need to be calculated for other measurements), method 700 loops back to 702 to select the next measurement and calculate p-values for all diagnosis tag pairs for that measurement. If the current measurement is the last measurement, method 700 ends. After calculations are performed for all pairs of tags and for all measurements associated with each pair of tags, p-values and any other included results from the calculations may be stored in a data structure which may be keyed by a pair of tags and ordered by p-values. In one example, the data structure may be ordered by p-values starting from a smallest p-value.

FIG. 8 shows a flow chart illustrating a method 800 for narrowing down a list of diagnosis tags to suggest to a user during a patient exam given a current set of measurements and a current set of diagnosis tags. In one example, method 800 may be executed during method 400 of FIG. 4 as a subroutine.

At 802, method 800 includes selecting two diagnosis tags from the list of diagnosis tags and obtaining the p-values for each measurement associated with the two diagnosis tags. A user may be presented with the current set of diagnosis tags on a display device of the system, and the system may iterate through any measurement not yet performed by the user to determine the lowest p-value of a measurement not yet performed based on the current set of diagnosis tags, as explained below.

At 804, method 800 includes suggesting the additional measurement with the lowest p-value to the user. The additional measurement with the lowest p-value may have a highest likelihood to narrow down the current set of diagnosis tags by at least one diagnosis tag. In one example, the additional measurement with the lowest p-value may have a highest likelihood to not be included in a typical range of one diagnosis tag in the current set of diagnosis tags, meaning that if the additional measurement is performed and the measurement value is not included in the typical range of one diagnosis tag in the current set of diagnosis tags, the diagnosis tag may be removed from the current set of diagnosis tags.

At 806, method 800 includes determining if an additional measurement is performed by the user. Any additional measurement performed may be considered in the determination (e.g., an additional measurement not suggested to the user). If the additional measurement is not performed, method 800 may end, or method 800 may loop back to 802 and may suggest a different measurement.

If the additional measurement is performed, method 800 may proceed to 808, which includes determining if the additional measurement performed includes a measurement value outside of a typical range for an associated diagnosis tag in the current set of diagnosis tags. If the additional measurement performed is the suggested measurement, there may be a higher likelihood that the measurement value of the additional measurement is not included in the typical range for the diagnosis tag included in the p-value calculation. If the measurement value of the additional measurement is not outside the typical range for an associated diagnosis tag in the current set of diagnosis tags, method 800 may proceed to 802 to determine a new lowest p-value of performing a new additional measurement given the current set of measurements and the current set of diagnosis tags.

If the measurement value of the additional measurement is outside of the typical range for an associated diagnosis tag in the current set of diagnosis tags, method 800 may proceed to 810, which includes removing the associated diagnosis tag from the set of displayed diagnosis tags. The user may be informed of any updates to the current set of diagnosis tags displayed on a display device of the system during the current patient exam by a notification and/or an indication of any removed diagnosis tags from the current set of diagnosis tags.

At 812, the above process may be repeated (e.g., determine a measurement with a lowest p-value that has yet to be performed to narrow down the possible diagnosis tags) until only a threshold number of diagnosis tags remain in the list of possible diagnosis tags or until additional measurements cannot be identified. For example, if the original list of suggested diagnosis tags is relatively long (e.g., 10 diagnosis tags) and the first suggested measurement only eliminates one diagnosis tag from the list, one or more additional measurements may be suggested until the list of suggested/possible diagnosis tags reaches the threshold, which may be one tag, two tags, or three tags. Alternatively, after suggesting one or more additional measurements, the system may not be able to identify further measurements that can differentiate between diagnosis tags. In either case (or upon the user indicating the exam is complete), method 800 may end.

By returning database queries when a set of measurements is received according to the processes described herein, several advantages may be achieved. The database and/or p-value datastructure may demand minimized memory usage, as only pertinent data from the exams may be saved (e.g., other information from the exams may be discarded) and the data may be structured in an efficient manner. When a query is performed to identify tags and/or measurements from a received set of measurements, the results may be returned quickly, particularly as the list of possible tags narrows with each additional measurement. Further, processing power may be reduced by providing for easy identification of relevant measurements and tag probability calculations, which may also be used to assist in identifying additional, diagnostically relevant measurements to be performed.

FIG. 9 shows an example process 900, where a plurality of possible diagnosis tags may be suggested based on one or more measurements, as well as additional measurements that may differentiate the suggested diagnosis tags. Process 900 may be one example illustrating how the relational database (or system executing the database) calculates probabilities of diagnosis tags being associated with the current patient exam to determine which measurements to suggest to the user and/or which diagnosis tags to suggest to the user, described above with respect to FIG. 4, but other approaches for making suggestions based on the database of measurements may be implemented without departing from the scope of the disclosure. In one example, all diagnosis tags that may be associated with the current patient exam given the current set of measurements may have probabilities calculated for each measurement in the database lookup in order to reduce the amount of diagnosis tags displayed to the user, allowing the user to make an accurate diagnosis with a minimum amount of measurements taken.

The process 900 may include an input 902, which may be an automatic query executed during a current patient exam when a user performs a measurement on an ultrasound image. In one example, input 902 may include a current set of measurements performed during the current patient exam. In the example shown in FIG. 9, the input includes a first measurement of intra ventricular septum diameter in 2D in diastole (IVSd) and a second measurement of left ventricular ejection fraction (EF), where the first measurement has a value of 12 mm and the second measurement has a value of 60%.

The input is entered into a database 904, which may include all known measurements, all known diagnosis tags, all prior patient exams, all typical ranges for all measurements and diagnosis tags, and the like. In one example, database 904 may include all possible p-values for each measurement and diagnosis tag pair.

As shown in FIG. 9, input 902 and database 904 may communicate via suggestion system 906, which may retrieve probable diagnosis tags and/or suggested measurements from database 904. However, in other examples, suggestion system 906 may also include a database of calculations for referencing any p-value calculations, mean value calculations, typical range calculations, and the like. Suggestion system 906 may take queries and/or inputs from input 902 as well as data (e.g., groupings, entries) from database 904 depending on the requested information in input 902. In one example, suggestion system 906 may be suggestion system 124 of FIG. 1. Suggestion system 906 may generate suggested diagnosis tags, suggested measurements, and/or the like based on the input from input 902, such that if input from input 902 is automatically generated by the system whenever the user performs an action like taking a measurement during a current patient exam, suggestion system 906 may automatically provide output after any user action is taken.

Suggestion system 906 may output suggestions 908, which may be suggested diagnosis tags, suggested measurements, and/or the like, to be displayed on a display device of the system. In one example, the current patient exam may include the same measurements as previous patient exams including a given diagnosis tag (e.g., tag X), so measurement values relating to the current patient exam may be compared to measurement values from previous exams including the given diagnosis tag, in order to determine if the current patient exam should also include the given diagnosis tag. When comparing measurement values, if a measurement value from the current patient exam is not in the range of values bounded by the typical range associated with the measurement, the given diagnosis tag may not be suggested by suggestion system 906. In this example, each measurement associated with a diagnosis tag (as determined by the database 904) may be present in the current exam, with values in predefined typical ranges, in order for that diagnosis tag to be suggested by suggestion system 906. However, in other examples, if a measurement is missing or has a measurement value outside a predefined typical range, the diagnosis tag may still be suggested by suggestion system 906, but with a lower confidence.

In another example, the current patient exam may not include a measurement that is included in previous patient exams that include a plurality of diagnosis tags currently associated with the current patient exam, so probabilities of each diagnosis tag being associated with the current patient exam after including the measurement may be compared for all diagnosis tags currently associated with the current patient exam that include the measurement currently not included in the current patient exam. If any probabilities of any diagnosis tags being associated with the current patient exam after including the measurement vary by an amount exceeding a variance threshold, it may be determined that the measurement may be suggested to the user by suggestion system 906 to narrow down a list of possible diagnosis tags to include in the current patient exam.

For example, the suggestions 908 may include a list of probable diagnosis tags determined based on the input 902. As shown, the probable diagnosis tags may include three tags: a first tag of normal, a second tag of primary aldosteronism, and a third tag of essential hypertension.

Because multiple, exclusive diagnosis tags are suggested in the list of probable diagnosis tags, one or more measurements may be suggested to narrow down the list of probable diagnosis tags. The suggested measurement(s) may be selected based on the p-values for each measurement and pair of diagnosis tags. In the example show, three pairs of diagnosis tags are possible (tag 1, tag2; tag 1, tag 3; and tag 2, tag 3). The p-values for each measurement for each of the three pairs of diagnosis tags may be retrieved from the p-value datastructure (which may be saved on database 904 and/or suggestion system 906) and the measurement having the lowest p-value from the retrieved p-values may be selected. As an example, FIG. 9 shows the p-values and associated measurements for one pair of diagnosis tags (tag 2, tag 3), which includes early diastolic annular velocity (e′) having a p-value of 0.004, iso volumetric relaxation time (IVRT) having a p-value of 0.28, and EF having a p-value of 0.94. The measurement with the lowest p-value that has not already been performed is then suggested, which in this case would be e′.

As explained previously, the suggested measurement may then be performed by the user and the value of the suggested measurement may be used to further narrow down the list of probable diagnosis tags. For example, if the user performed the e′ measurement and determined it had a value of 8 cm/s, the measured value of 8 cm/s may be entered as an input and compared to the typical range for the e′ measurement for each of the probable diagnosis tags. If the value is outside the typical range for any of the probable diagnosis tags, that tag may be excluded. For example, the typical range for e′ for primary aldosteronism may be 5.8+/−1.9, and thus tag 2 may be excluded based on the value of the suggested measurement. The process may then be repeated to suggest a measurement to differentiate between tag 1 and tag 3.

One mechanism for suggesting additional measurements to narrow down or differentiate among possible tags includes generating a database with the summed conditional probability for each tag iteratively recalculated with a new (not yet performed) measurement included. For example, if in the example presented above, a first measurement was not performed, but the first measurement is found in at least some exams with a first tag and/or a second tag, the summed conditional probability for each of the first tag and the second tag may be recalculated with the respective conditional probabilities for the first measurement included in the calculation. If the summed probability increases for one tag more than the other tag, the first measurement may be suggested to the user by suggestion system 906 for a measurement to be performed to differentiate the second tag from the first tag. In another example, if a second measurement is more highly correlated with the second diagnosis tag than the first diagnosis tag, and thus if the second measurement has not been performed yet, it may be suggested to the user (or another measurement that is more highly correlated with one of the first diagnosis tag or the second diagnosis tag than the other and has yet to be performed) by suggestion system 906.

In another example, if the analysis of the measurements described above returned two possible diagnosis tags with substantially equal probability, in order to determine if a given measurement could distinguish between the two diagnosis tags, a further analysis of the data in the database 904 may be performed by suggestion system 906. For example, the prior patient exams tagged with either a first tag or a second tag may be selected, and the values for a first measurement may be plotted as a function of the values for a second measurement associated with the first tag and the second tags. If the exams do not include measurements with p-values to discern the first tag from the second tag based on the value of the first measurement, then the first measurement may be determined to not be valuable for discriminating the first tag from the second tag. For example, if the exams have different measurement values for the second measurement but all the same (or relatively same) measurement values for the first measurement, then the first measurement may be determined to not be discriminating. On the other hand, if the exams do include measurements with p-values to discern the first tag from the second tag based on the first measurement (e.g., the exams that have the second tag all have values for the first measurement above a threshold value and the exams that have the first tag all have values for the first measurement below the threshold value), the first measurement may be determined to be valuable for discriminating the first tag from the second tag and thus suggested to the user by suggestion system 906.

As another example of how to determine if a measurement will aid in differentiating two possible diagnosis tags, the probability of a measurement value of the first measurement being associated with a first possible diagnosis tag may be plotted as a function of the measurement values for that measurement. Similarly, the probability of a measurement value of the second measurement being associated with the second possible diagnosis tag may be plotted as a function of the measurement values for that measurement. The distribution of the probabilities may then be compared to determine if that measurement may be valuable in discriminating the two diagnosis tags. For example, if the probability curves are similar and/or have high overlap (e.g., peak at the same measurement value and/or encompass the same measurement values), that measurement may be determined not to be valuable. However, if the probability curves are not similar and/or do not have a high amount of overlap (e.g., the peaks of the curves are offset by a certain amount), then it may be determined that the measurement is valuable for discriminating the two diagnosis tags and suggested to the user.

While the suggestion system, database of measurements, and methods for calculating typical ranges of the data and suggesting possible diagnoses and measurements to a user have been described herein with respect to an ultrasound system, it is to be appreciated that the systems and methods described herein may be applied to other types of medical images, including but not limited to magnetic resonance images, computed tomography images, X-ray images, visible light images, and the like.

A technical effect of suggesting possible diagnoses and/or measurements for a patient exam, based on one or more measurements of anatomical features in medical images of the patient, using a database of measurements is that accurate diagnoses may be provided and/or a level of confidence in a diagnosis may be increased, improving patient care. A further technical effect is that the database of measurements may be implemented on a variety of devices and allow for suggested diagnoses and/or measurements in a variety of clinical settings, while reducing the processing power and memory demanded of other computer-aided diagnosis systems.

The disclosure also provides support for a method for a user interface of a medical imaging system, comprising: receiving a first user input from a user and determining a first measurement of a medical image based on the first user input, sending the first measurement and a value of the first measurement to a database of measurements, receiving, from the database, a second suggested measurement determined based on diagnosis tags each including a typical range for the first measurement including the value of the first measurement, and suggesting the second suggested measurement to the user before the user performs the second measurement. In a first example of the method, each typical range is a range of values for the first measurement included in the database of measurements and associated with a respective diagnosis tag. In a second example of the method, optionally including the first example, each typical range is based on a mean value and a standard deviation of all values for the first measurement included in the database of measurements and associated with a respective diagnosis tag. In a third example of the method, optionally including one or both of the first and second examples, the method further comprises: receiving a second user input from the user and determining the second measurement of the medical image based on the second user input, sending the second measurement and a value of the second measurement to the database of measurements, receiving, from the database, a third suggested measurement determined based on diagnosis tags each including the typical range for the first measurement including the value of the first measurement and a second typical range for the second measurement including the value of the second measurement, and suggesting the third suggested measurement to the user before the user performs the third measurement. In a fourth example of the method, optionally including one or more or each of the first through third examples, the method further comprises: receiving a second user input from the user and determining the second measurement of the medical image based on the second user input, sending the second measurement and a value of the second measurement to the database of measurements, receiving, from the database, a suggested first diagnosis tag indicating a possible diagnosis code or finding based on the value of the first measurement and the value of the second measurement, and displaying the suggested first diagnosis tag. In a fifth example of the method, optionally including one or more or each of the first through fourth examples, upon receiving the first measurement and the value of the first measurement, the database identifies the first diagnosis tag and a second diagnosis tag, and wherein the second measurement is suggested to differentiate the first diagnosis tag and the second diagnosis tag. In a sixth example of the method, optionally including one or more or each of the first through fifth examples, the second measurement is suggested by obtaining a list of measurements and associated p-values from the database and selecting a measurement from the list of measurements that has a lowest p-value, wherein the second measurement is the selected measurement. In a seventh example of the method, optionally including one or more or each of the first through sixth examples, each p-value indicates a level of statistical difference between the first diagnosis tag and the second diagnosis tag based on the corresponding measurement. In an eighth example of the method, optionally including one or more or each of the first through seventh examples, the medical image is of a patient, and further comprising, responsive to receiving a third user input selecting the first diagnosis tag, saving the first diagnosis tag as part of a report for the patient, the report further including the medical image, the first measurement, the value of the first measurement, the second measurement, and the value of the second measurement. In a ninth example of the method, optionally including one or more or each of the first through eighth examples, the medical image is of a patient, and further comprising displaying one or more additional diagnosis tags and responsive to a third user input selecting a diagnosis tag of the one or more additional diagnosis tags, saving the selected diagnosis tag as part of a report for the patient, the report further including the medical image, the first measurement, the value of the first measurement, the second measurement, and the value of the second measurement. In a tenth example of the method, optionally including one or more or each of the first through ninth examples, upon receiving the first measurement and the value of the first measurement, the database identifies a first subset of possible diagnosis tags from among a plurality of possible diagnosis tags listed in the database and excludes all other diagnosis tags in the plurality of possible diagnosis tags based on diagnosis tags including typical ranges including the value of the first measurement, upon receiving the second measurement and the value of the second measurement, the database identifies a second subset of possible diagnosis tags from among the first subset of possible diagnosis tags and excludes all other diagnosis tags in the first subset of diagnosis tags based on diagnosis tags including typical ranges including the value of the first measurement and the value of the second measurement, and further comprising receiving the second subset of diagnosis tags from the database and displaying the second subset of diagnosis tags.

The disclosure also provides support for a method, comprising: receiving a first measurement of an anatomical feature in a medical image and a value of the first measurement, mapping the first measurement and the value of the first measurement to at least a first diagnosis tag and a second diagnosis tag via a database of measurements based on the value of the first measurement falling within a respective typical range of values for the first measurement for each of the first diagnosis tag and the second diagnosis tag, each diagnosis tag indicative of a diagnosis code or finding relating to the anatomical feature in the medical image, and determining that a second measurement of the anatomical feature will differentiate between the first diagnosis tag and the second diagnosis tag without clustering the tags and measurements, and in response, outputting a suggestion that the second measurement should be performed. In a first example of the method, the typical range of values for the first measurement for the first diagnosis tag is based on a mean value and a standard deviation of all values for the first measurement included in the database of measurements and associated with the first diagnosis tag, and wherein the typical range of values for the first measurement for the second diagnosis tag is based on a mean value and a standard deviation of all values for the first measurement included in the database of measurements and associated with the second diagnosis tag. In a second example of the method, optionally including the first example, determining that the second measurement of the anatomical feature will differentiate between the first diagnosis tag and the second diagnosis tag includes determining that the second measurement has a lowest associated p-value from among a list of possible measurements and associated p-values. In a third example of the method, optionally including one or both of the first and second examples, the database of measurements includes a database of p-values, each p-value calculated for a given measurement and between a pair of diagnosis tags based on a first typical range of values for the given measurement for a first diagnosis tag of the pair of diagnosis tags and a second typical range of values for the given measurement for a second diagnosis tag of the pair of diagnosis tags, and wherein determining that the second measurement has the lowest associated p-value from among the list of possible measurements and associated p-values includes obtaining the list of possible measurements and associated p-values from the database of p-values. In a fourth example of the method, optionally including one or more or each of the first through third examples, the database of measurements comprises data from a plurality of prior patient exams. In a fifth example of the method, optionally including one or more or each of the first through fourth examples, the data in the database of measurements includes data from only prior patient exams that include less than a first threshold number of diagnosis tags, a number of measurements within a second threshold range, and an indication that the prior patient exam is complete. In a sixth example of the method, optionally including one or more or each of the first through fifth examples, the one or more diagnosis tags in each exam comprise user-defined tags and non-user-defined tags, and wherein only consistently-used user-defined tags are included in the database of measurements, wherein consistently-used user-defined tags are identified based on a frequency of usage and/or time period of usage.

The disclosure also provides support for a system, comprising: a memory storing instructions, and a processor configured to execute the instructions to: receive a set of measurements of patient anatomical features present in one or more medical images of a patient, each measurement having a respective value, identify, using a database of measurements generated from a plurality of prior exams, a list of diagnosis tags based on the set of measurements and respective value of each measurement, where each diagnosis tag in the list of diagnosis tags is identified from the database of measurements based on each diagnosis tag in the list of diagnosis tags being associated with, in the database of measurements, each measurement of the set of measurements, including each diagnosis tag in the list of diagnosis tags having, for each measurement of the set of measurements, a respective typical range of values that spans the corresponding respective value, each diagnosis tag in the list of diagnosis tags indicating a respective diagnosis code or finding, the list of diagnosis tags identified from among a plurality of possible diagnosis tags from the database of measurements, and output the list of diagnosis tags for display on a display device. In a first example of the system, the processor is further configured to execute the instructions to: identify an additional measurement to differentiate between a first diagnosis tag and a second diagnosis tag of the list of diagnosis tags based on a list of measurements and associated p-values from the database of measurements, and output a suggestion to perform the additional measurement for display on the display device, and wherein each typical range is a range of values for a respective measurement associated with a respective diagnosis tag, each typical range based on a spread of values determined from all measurement values for that measurement associated with that diagnosis tag in the database of measurements.

When introducing elements of various embodiments of the present disclosure, the articles “a,” “an,” and “the” are intended to mean that there are one or more of the elements. The terms “first,” “second,” and the like, do not denote any order, quantity, or importance, but rather are used to distinguish one element from another. The terms “comprising,” “including,” and “having” are intended to be inclusive and mean that there may be additional elements other than the listed elements. As the terms “connected to,” “coupled to,” etc. are used herein, one object (e.g., a material, element, structure, member, etc.) can be connected to or coupled to another object regardless of whether the one object is directly connected or coupled to the other object or whether there are one or more intervening objects between the one object and the other object. In addition, it should be understood that references to “one embodiment” or “an embodiment” of the present disclosure are not intended to be interpreted as excluding the existence of additional embodiments that also incorporate the recited features.

In addition to any previously indicated modification, numerous other variations and alternative arrangements may be devised by those skilled in the art without departing from the spirit and scope of this description, and appended claims are intended to cover such modifications and arrangements. Thus, while the information has been described above with particularity and detail in connection with what is presently deemed to be the most practical and preferred aspects, it will be apparent to those of ordinary skill in the art that numerous modifications, including, but not limited to, form, function, manner of operation and use may be made without departing from the principles and concepts set forth herein. Also, as used herein, the examples and embodiments, in all respects, are meant to be illustrative only and should not be construed to be limiting in any manner.

1. A method for a user interface of a medical imaging system, comprising:
receiving a first user input from a user and determining a first measurement of a medical image based on the first user input;
sending the first measurement and a value of the first measurement to a database of measurements;
receiving, from the database, a suggested second measurement determined based on diagnosis tags each including a typical range for the first measurement including the value of the first measurement; and
suggesting the second suggested measurement to the user before the user performs the second measurement.

2. The method of claim 1, wherein each typical range is a range of values for the first measurement included in the database of measurements and associated with a respective diagnosis tag.

3. The method of claim 2, wherein each typical range is based on a mean value and a standard deviation of all values for the first measurement included in the database of measurements and associated with a respective diagnosis tag.

4. The method of claim 1 further comprising:
receiving a second user input from the user and determining the second measurement of the medical image based on the second user input;
sending the second measurement and a value of the second measurement to the database of measurements;
receiving, from the database, a suggested third measurement determined based on diagnosis tags each including the typical range for the first measurement including the value of the first measurement and a second typical range for the second measurement including the value of the second measurement; and
suggesting the third measurement to the user before the user performs the third measurement.

5. The method of claim 1, further comprising:
receiving a second user input from the user and determining the second measurement of the medical image based on the second user input;
sending the second measurement and a value of the second measurement to the database of measurements;
receiving, from the database, a suggested first diagnosis tag indicating a possible diagnosis code or finding based on the value of the first measurement and the value of the second measurement; and
displaying the suggested first diagnosis tag.

6. The method of claim 5, wherein upon receiving the first measurement and the value of the first measurement, the database identifies the first diagnosis tag and a second diagnosis tag, and wherein the second measurement is suggested to differentiate the first diagnosis tag and the second diagnosis tag.

7. The method of claim 6, wherein the second measurement is suggested by obtaining a list of measurements and associated p-values from the database and selecting a measurement from the list of measurements that has a lowest p-value, wherein the second measurement is the selected measurement.

8. The method of claim 7, wherein each p-value indicates a level of statistical difference between the first diagnosis tag and the second diagnosis tag based on the associated measurement.

9. The method of claim 5, wherein the medical image is of a patient, and further comprising, responsive to receiving a third user input selecting the first diagnosis tag, saving the first diagnosis tag as part of a report for the patient, the report further including the medical image, the first measurement, the value of the first measurement, the second measurement, and the value of the second measurement.

10. The method of claim 5, wherein the medical image is of a patient, and further comprising displaying one or more additional diagnosis tags and responsive to a third user input selecting a diagnosis tag of the one or more additional diagnosis tags, saving the selected diagnosis tag as part of a report for the patient, the report further including the medical image, the first measurement, the value of the first measurement, the second measurement, and the value of the second measurement.

11. The method of claim 5, wherein upon receiving the first measurement and the value of the first measurement, the database identifies a first subset of possible diagnosis tags from among a plurality of possible diagnosis tags listed in the database and excludes all other diagnosis tags in the plurality of possible diagnosis tags based on diagnosis tags including typical ranges including the value of the first measurement;
upon receiving the second measurement and the value of the second measurement, the database identifies a second subset of possible diagnosis tags from among the first subset of possible diagnosis tags and excludes all other diagnosis tags in the first subset of diagnosis tags based on diagnosis tags including typical ranges including the value of the first measurement and the value of the second measurement; and
further comprising receiving the second subset of diagnosis tags from the database and displaying the second subset of diagnosis tags.

12. A method, comprising:
receiving a first measurement of an anatomical feature in a medical image and a value of the first measurement;
mapping the first measurement and the value of the first measurement to at least a first diagnosis tag and a second diagnosis tag via a database of measurements based on the value of the first measurement falling within a respective typical range of values for the first measurement for each of the first diagnosis tag and the second diagnosis tag, each diagnosis tag indicative of a diagnosis code or finding relating to the anatomical feature in the medical image; and
determining that a second measurement of the anatomical feature will differentiate between the first diagnosis tag and the second diagnosis tag without clustering the tags and measurements, and in response, outputting a suggestion that the second measurement should be performed.

13. The method of claim 12, wherein the typical range of values for the first measurement for the first diagnosis tag is based on a mean value and a standard deviation of all values for the first measurement included in the database of measurements and associated with the first diagnosis tag, and wherein the typical range of values for the first measurement for the second diagnosis tag is based on a mean value and a standard deviation of all values for the first measurement included in the database of measurements and associated with the second diagnosis tag.

14. The method of claim 12, wherein determining that the second measurement of the anatomical feature will differentiate between the first diagnosis tag and the second diagnosis tag includes determining that the second measurement has a lowest associated p-value from among a list of possible measurements and associated p-values.

15. The method of claim 14, wherein the database of measurements includes a database of p-values, each p-value calculated for a given measurement and between a pair of diagnosis tags based on a first typical range of values for the given measurement for a first diagnosis tag of the pair of diagnosis tags and a second typical range of values for the given measurement for a second diagnosis tag of the pair of diagnosis tags, and wherein determining that the second measurement has the lowest associated p-value from among the list of possible measurements and associated p-values includes obtaining the list of possible measurements and associated p-values from the database of p-values.

16. The method of claim 12, wherein the database of measurements comprises data from a plurality of prior patient exams.

17. The method of claim 16, wherein the data in the database of measurements includes data from only prior patient exams that include less than a first threshold number of diagnosis tags, a number of measurements within a second threshold range, and an indication that the prior patient exam is complete.

18. The method of claim 17, wherein the one or more diagnosis tags in each exam comprise user-defined tags and non-user-defined tags, and wherein only consistently-used user-defined tags are included in the database of measurements, wherein consistently-used user-defined tags are identified based on a frequency of usage and/or time period of usage.

19. A system, comprising:
a memory storing instructions; and
a processor configured to execute the instructions to:
receive a set of measurements of patient anatomical features present in one or more medical images of a patient, each measurement having a respective value;
identify, using a database of measurements generated from a plurality of prior exams, a list of diagnosis tags based on the set of measurements and respective value of each measurement, where each diagnosis tag in the list of diagnosis tags is identified from the database of measurements based on each diagnosis tag in the list of diagnosis tags being associated with, in the database of measurements, each measurement of the set of measurements, including each diagnosis tag in the list of diagnosis tags having, for each measurement of the set of measurements, a respective typical range of values that spans the corresponding respective value, each diagnosis tag in the list of diagnosis tags indicating a respective diagnosis code or finding, the list of diagnosis tags identified from among a plurality of possible diagnosis tags from the database of measurements; and
output the list of diagnosis tags for display on a display device.

20. The system of claim 19, wherein the processor is further configured to execute the instructions to:
identify an additional measurement to differentiate between a first diagnosis tag and a second diagnosis tag of the list of diagnosis tags based on a list of measurements and associated p-values from the database of measurements; and
output a suggestion to perform the additional measurement for display on the display device, and wherein each typical range is a range of values for a respective measurement associated with a respective diagnosis tag, each typical range based on a spread of values determined from all measurement values for that measurement associated with that diagnosis tag in the database of measurements.
